(19) United States
(12) Patent Application Publication
(10) Pub. No.: US 2010/0071086 A1
(43) Pub. Date: Mar. 18, 2010

(54) POLYSOME-MEDIATED CELL TYPE-, TISSUE TYPE- OR CONDITION-ENHANCED TRANSCRIPT PROFILING

(75) Inventors: Peter P. Repetti, Emeryville, CA (US); Oliver J. Ratcliffe, Oakland, CA (US); Luc J. Adam, Hayward, CA (US); T. Lynne Reuber, San Mateo, CA (US); Hans E. Holtan, Emeryville, CA (US)

Correspondence Address: MENDEL 2 C/O MOFO SF, 425 MARKET STREET, SAN FRANCISCO, CA, 94105, US

(73) Assignee: MENDEL BIOTECHNOLOGY, INC., Hayward, CA (US)

(21) Appl. No.: 12/557,449

(22) Filed: Sep. 10, 2009

Related U.S. Application Data
(60) Provisional application No. 61/096,708, filed on Sep. 12, 2008.

(51) Int. Cl. C12N 15/82; A01H 5/00; C12N 5/04; C12N 15/74

(52) U.S. Cl. 800/278; 800/295; 435/410; 435/320.1

(57) ABSTRACT

In this invention, a method is described that allows for the efficient creation and identification of validated biological materials that greatly enhance the ability to perform polysome-mediated RNA profiling, such as constitutive, cell type-, tissue type-, or condition-enhanced RNA profiling. The method relies on the use of a tri-partite plant binary expression vector comprised of the following components: a) a DNA promoter element that drives expression of a sequence specific transcription activator protein such as a LexA:Gal4 fusion protein in a unique desired pattern, b) a DNA promoter element comprising a target site for the transcriptional activator protein, such as opLexA, fused to a nucleotide encoding an epitope tagged ribosomal component protein and c) a DNA promoter element comprising a target site for the transcriptional activator protein, such as opLexA, fused to a nucleotide encoding an in vivo reporter protein. By visualization of the co-regulated reporter, this method allows for in planta confirmation that the promoter element is driving expression, such as constitutive, cell type-, tissue type-, or condition-enhanced expression, of the tagged ribosomal protein in the desired cell or tissue types.

### CROSS REFERENCE TO RELATED APPLICATIONS

This application claims priority to U.S. Provisional Patent Application Ser. No. 61/096,708, filed on Sep. 12, 2008, which is hereby incorporated by reference in its entirely.

### FIELD OF THE INVENTION

The present invention relates to plant genomics and plant improvement, and systems biology.

### BACKGROUND OF THE INVENTION

As the field of Systems Biology develops in multicellular organisms, it is critical to gain insight into processes occurring in distinct tissues and cell types. However, the biology at the organismal level can only be fully appreciated by an understanding of the local responses in specific groups of cells. Techniques such as expression profiling, often used to generate ‘Systems’ data, are confounded by the very nature of multicellularity. That is, key regulatory signals and responses that occur in the small groups of cells that make up specific tissues are diluted by the background of downstream responses or the otherwise steady state of more abundant cells.

A variety of approaches have been developed in an effort to enable the researcher's ability to obtain such data from specific cell types. These techniques invariably disrupt or change the basic multicellular nature that is the ultimate object of study (e.g., protoplasting followed by cell sorting), or are extremely labor intensive and require dedicated equipment (e.g., laser microdissection). Recently, an approach has been devised for capturing RNA transcripts actively translated in specific cell types via a small-epitope tagged ribosomal protein (e.g., RPL18) driven by a cell-specific promoter fragment (Zanetti et al. (2005) Plant Physiol. 138: 624-635). A DNA construct encoding this protein ‘tool’ could be transformed into the organism of interest, and the polysomes with associated RNA from the promoter-specified cell types would then be recovered by performing immunoprecipitation against the epitope tag. Such recovered RNA could then be used in downstream applications such as transcript profiling by microarray or high throughput sequencing.

To perform the type of experiment outlined above, a well-characterized promoter fragment that drives gene expression in a specific cell type or condition is a key requirement. However, unintended “position effects” at the location of the transgene insertion often alter the expression pattern derived from the transgenic promoter and thereby result in expression of the tagged ribosomal protein in unintended patterns. This problem presents one of the most challenging aspects of such work; in order to obtain a reliable transgenic line for use in RNA pull down experiments from specific cells or tissues, a multitude of independent lines need to be laboriously screened by in situ RNA hybridization or immunohybridization via the epitope tag to obtain a line in which the tagged ribosomal protein is expressed in the desired pattern

The method presented here represents an improvement to previous approaches and enables very efficient screening and selection of transgenic events expressing epitope-tagged polysomes in specific cell or tissue types via a single vector “multi-component” reporter system.

### SUMMARY OF THE INVENTION

This invention comprises a method for enhancing the ability to perform polysome-mediated cell-specific RNA profiling. The method is practiced with a single vector “multi-component” system in order to express an epitope-tagged ribosomal protein and a reporter protein that are co-regulated by a single promoter driving expression in a desired cell-, tissue-, or condition-enhanced pattern. The first component of the vector comprises the desired promoter cloned in front of a sequence encoding a specific transcriptional activator protein whereas the second component comprises a DNA target site to which the transcriptional activator protein will bind in a sequence specific manner. The latter target site is separately fused to both the epitope tagged ribosomal protein and to the reporter protein. In the example detailed here, the transformation vector carries a first component corresponding to a promoter fused to a sequence encoding a LexA DNA binding domain fused to a GAL4 activation domain, but any other known DNA binding/activation sequence could be used. Additionally, the vector contains a second component nucleic acid sequence encoding the opLexA DNA target site (to which the LexA-GAL4 fusion binds and activates transcription) fused to the RPL18 ribosomal protein (AT3G05590) coding sequence with a 6×HIS-FLAG epitope coding region at the 5′ end. A variety of other common protein epitope tags such as cMyc, HA (hemagglutinin), etc., could be used with the RPL18 ribosomal protein or any other ribosomal subunit protein, such as RPL12 (AT3G27830) or RPL23a (AT2G39460). The final component nucleic acid sequence encodes the opLexA::reporter (e.g., green fluorescent protein GFP). The construct may also encode at least one antibiotic resistance marker for selection of transgenic events, and the other required components for transfer and integration into the desired organism.

The invention also pertains to a nucleic acid construct, a host cell transformed with and comprising said construct, or a plant transformed with and comprising said construct, wherein the nucleic acid construct comprises the single vector “multi-component” system described above in this summary.

### BRIEF DESCRIPTION OF THE SEQUENCE LISTING AND DRAWINGS

The Sequence Listing provides exemplary polynucleotide and polypeptide sequences of the invention. The traits associated with the use of the sequences are included in the Examples.

Incorporation of the Sequence Listing. The copy of the Sequence Listing, being submitted electronically with this patent application, provided under 37 CFR §1.821-1.825, is a read-only memory computer-readable file in ASCII text format. The Sequence Listing is named “MBI-0085P_ST25.txt”, the electronic file of the Sequence Listing was created on Sep. 9, 2008, and is 131 kilobytes in size (measured in MS-WINDOWS). The Sequence Listing is herein incorporated by reference in its entirety.

FIG. 1 shows simplified (A-C) and detailed (D) schematics illustrating the relevant components of a plant transformation vector required for the present invention. Shown are three open reading frames: A. the desired cell-, tissue-, or condition-enhanced promoter fragment controlling the LexA-Gal4 fusion protein, B. the LexA-Gal4-controlled epitope-tagged ribosomal protein (e.g., HFRPL18=his/FLAG epitope N-terminally fused to the RPL18 ribosomal protein, SEQ ID NO: 2) and C. the LexA-Gal4-controlled reporter (e.g., GFP). The plasmid map in FIG. 1D shows SEQ ID NO: 1, an example of a base vector with, in addition to the components in A-C, features such as a multi-cloning site to insert desired promoters (MCS), terminator sequences (rubisco E9 (e9) and octopine synthase (ocs), and a plant-based selectable marker (Kan=kanamycin).

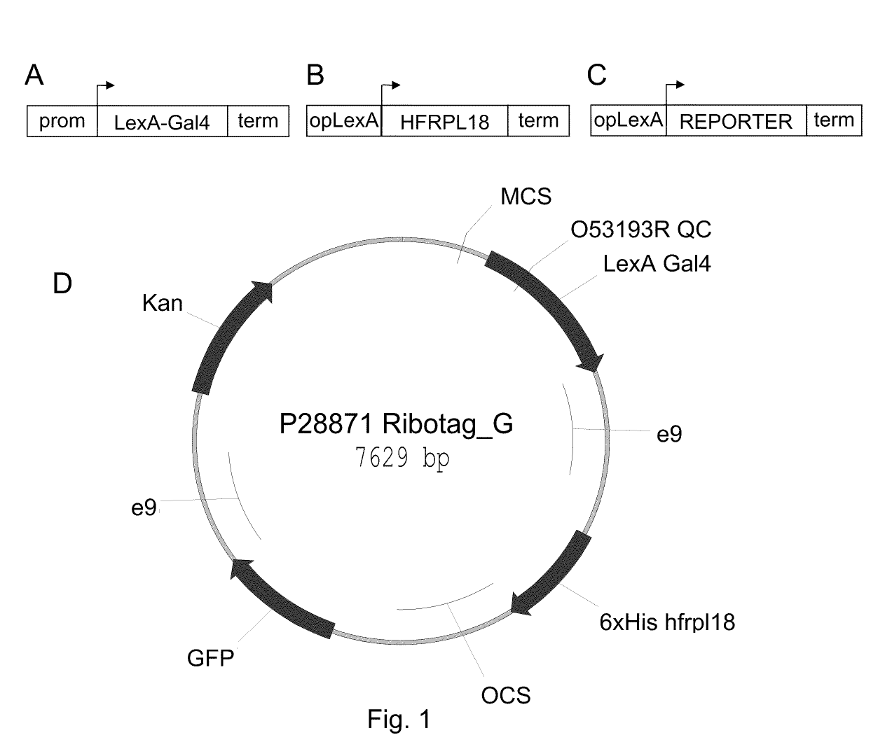

FIG. 2 shows representative opLexA::GFP expression patterns of exemplary promoters. In FIG. 2A (pattern derived with prSUC2, SEQ ID NO: 9), the arrows indicate primary and secondary vascular tissue, in FIG. 2B (pattern derived with prG682, SEQ ID NO: 18) the arrows indicate guard cells, and in FIG. 2C (pattern derived with prRBCS1A, SEQ ID NO: 10, the arrow indicates the root/shoot (green tissue) boundary.

FIG. 3. Enrichment of vascular mRNA in prSUC2::RiboTag immunoprecipitations. mRNA recovered from immunoprecipitations performed on either 35S::RiboTag or prSUC2::RiboTag plant lines (23 days old) was subjected to qPCR for either negative control genes (enrichment of GC1 (AT1G22690; SEQ ID NO: 5) and AT2G01520 (MLP328, SEQ ID NO: 4) shown in FIGS. 3A and 3B, respectively) or positive control genes (enrichment of SUC2 (SEQ ID NO: 8) and AHA3 (AT5G57350, SEQ ID NO: 6) shown in FIGS. 3C and 3D, respectively). GC1 and AT2G01520 are guard cell or root-specific transcripts, respectively, and do not show enriched abundance in vascular prSUC2-specified cell types. SUC2 and AHA3 transcripts are known in the literature to be localized to vascular tissue, and were found to be enriched >100-fold in prSUC2::RiboTag tissue. The y-axis shows the ratio obtained by comparing the PCR cycle threshold count of the RiboTag sample vs. the control (in this case, the 35S sample). Results from two independent biological experiments are shown (rep 1 and rep 2).

FIG. 4. Enrichment of guard cell mRNA in prG682::RiboTag immunoprecipitations. mRNA recovered from immunoprecipitations performed on either 35S::RiboTag or prG682::RiboTag plant lines (23 days old) was subjected to qPCR for either negative control genes (enrichment of SUC2 (SEQ ID NO: 8) and AT2G01520 (MLP328, SEQ ID NO: 4) shown in FIGS. 4A and 4B, respectively) or positive control genes (enrichment of GC1 (AT1G22690, SEQ ID NO: 5) and KAT1 (AT5G46240, SEQ ID NO: 7), shown in FIGS. 4C and 4D, respectively). SUC2 and AT2G01520 are vascular or root-specific transcripts, respectively, and do not show enriched abundance in guard cell prG682-specified cell types. GC1 and KAT1 transcripts are known in the literature to be localized to guard cells, and were found to be enriched 20-fold or 4-fold, respectively, in prG682::RiboTag tissue. The y-axis shows the ratio obtained by comparing the PCR cycle threshold count of the RiboTag sample vs. the control (in this case, the 35S sample). Results from two technical replicate experiments are shown (rep 1 and rep 2).

### DETAILED DESCRIPTION OF THE INVENTION

The present invention relates to polynucleotides and polypeptides for improving cell-, tissue-, or condition-enhanced RNA profiling. Throughout this disclosure, various information sources are referred to and/or are specifically incorporated. The information sources include scientific journal articles, patent documents, textbooks, and World Wide Web browser-inactive page addresses. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the invention.

As used herein and in the appended claims, the singular forms “a”, “an”, and “the” include the plural reference unless the context clearly dictates otherwise. Thus, for example, a reference to “a host cell” includes a plurality of such host cells, and a reference to “a stress” is a reference to one or more stresses and equivalents thereof known to those skilled in the art, and so forth.

### DEFINITIONS

“Polynucleotide” is a nucleic acid molecule comprising a plurality of polymerized nucleotides, for example, at least about 15 consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone. The polynucleotide can be, for example, genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single-stranded.

A “recombinant polynucleotide” is a polynucleotide that is not in its native state, for example, the polynucleotide comprises a nucleotide sequence not found in nature, or the polynucleotide is in a context other than that in which it is naturally found, for example, separated from nucleotide sequences with which it typically is in proximity in nature, or adjacent (or contiguous with) nucleotide sequences with which it typically is not in proximity. For example, the sequence at issue can be cloned into a nucleic acid construct, or otherwise recombined with one or more additional nucleic acid.

An “isolated polynucleotide” is a polynucleotide, whether naturally occurring or recombinant, that is present outside the cell in which it is typically found in nature, whether purified or not. Optionally, an isolated polynucleotide is subject to one or more enrichment or purification procedures, for example, cell lysis, extraction, centrifugation, precipitation, or the like.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as chemical modification or folding to obtain a functional protein or polypeptide. A gene may be isolated, partially isolated, or found with an organism's genome.

Operationally, genes may be defined by the cis-trans test, a genetic test that determines whether two mutations occur in the same gene and that may be used to determine the limits of the genetically active unit (Rieger et al. (1976) Glossary of Genetics and Cytogenetics: Classical and Molecular, 4th ed., Springer Verlag, Berlin). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “polypeptide” is an amino acid sequence comprising a plurality of consecutive polymerized amino acid residues for example, at least about 15 consecutive polymerized amino acid residues. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, for example, more than about 5% enriched, more than about 10% enriched, or more than about 20%, or more than about 50%, or more, enriched, that is, alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, for example, by any of the various protein purification methods herein.

“Fragment”, with respect to a polynucleotide, refers to a clone or any part of a polynucleotide molecule that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about 9 consecutive nucleotides, preferably at least about 30 nucleotides, more preferably at least about 50 nucleotides, of any of the sequences provided herein. Exemplary polynucleotide fragments are the first sixty consecutive nucleotides of the polynucleotides listed in the Sequence Listing. Exemplary fragments also include fragments that comprise a region that encodes an conserved domain of a polypeptide. Exemplary fragments also include fragments that comprise a conserved domain of a polypeptide.

Fragments may also include subsequences of polypeptides and protein molecules, or a subsequence of the polypeptide. Fragments may have uses in that they may have antigenic potential. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, but are preferably at least about 30 amino acid residues in length and more preferably at least about 60 amino acid residues in length.

The invention also encompasses production of DNA sequences that encode polypeptides and derivatives, or fragments thereof, entirely by synthetic chemistry. After production, the synthetic sequence may be inserted into any of the many available nucleic acid constructs and cell systems using reagents well known in the art. Moreover, synthetic chemistry may be used to introduce mutations into a sequence encoding polypeptides or any fragment thereof.

The term “plant” includes whole plants, shoot vegetative organs/structures (for example, leaves, stems, rhizomes, and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like), calli, protoplasts, and cells (for example, guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, multicellular algae, and unicellular algae.

A “control plant” as used in the present invention refers to a plant cell, seed, plant component, plant tissue, plant organ or whole plant used to compare against transformed, transgenic or genetically modified plant for the purpose of identifying an enhanced phenotype in the transformed, transgenic or genetically modified plant. A control plant may in some cases be a transformed or transgenic plant line that comprises an empty nucleic acid construct or marker gene, but does not contain the recombinant polynucleotide of the present invention that is expressed in the transformed, transgenic or genetically modified plant being evaluated. In general, a control plant is a plant of the same line or variety as the transformed, transgenic or genetically modified plant being tested. A suitable control plant would include a genetically unaltered or non-transgenic plant of the parental line used to generate a transformed or transgenic plant herein.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a polypeptide's expression is altered, for example, in that it has been knocked out, overexpressed, or ectopically expressed.

“Transformation” refers to the transfer of a foreign polynucleotide sequence into the genome of a host organism such as that of a plant or plant cell, or introduction of a foreign polynucleotide sequence into plant or plant cell such that is expressed and results in production of protein. Typically, the foreign genetic material has been introduced into the plant by human manipulation, but any method can be used as one of skill in the art recognizes. Examples of methods of plant transformation include Agrobacterium-mediated transformation (De Blaere et. al. (1987) Meth. Enzymol., vol. 153: 277-292) and biolistic methodology (U.S. Pat. No. 4,945,050 to Klein et al.).

A “transformed plant”, which may also be referred to as a “transgenic plant” or “transformant”, generally refers to a plant, a plant cell, plant tissue, seed or calli that has been through, or is derived from a plant cell that has been through, a stable or transient transformation process in which a “nucleic acid construct” that contains at least one exogenous polynucleotide sequence is introduced into the plant. The “nucleic acid construct” contains genetic material that is not found in a wild-type plant of the same species, variety or cultivar, or may contain extra copies of a native sequence under the control of its native promoter. In some embodiments the a nucleic acid sequence transformed into a plant may be derived from the host plant, but by its incorporation into a nucleic acid construct, represents an element not found in a wild-type plant of the same species, variety or cultivar.

An “untransformed plant” is a plant that has not been through the transformation process.

A “stably transformed” plant, plant cell or plant tissue has generally been selected and regenerated on a selection media following transformation.

A “nucleic acid construct” may comprise a polypeptide-encoding sequence operably linked (that is, under regulatory control of) to appropriate inducible, cell-specific, tissue-specific, cell-enhanced, tissue-enhanced, condition-enhanced, developmental, or constitutive regulatory sequences that allow for the controlled expression of polypeptide. The expression vector or cassette can be introduced into a plant by transformation or by breeding after transformation of a parent plant. A plant refers to a whole plant as well as to a plant part, such as seed, fruit, leaf, or root, plant tissue, plant cells or any other plant material, for example, a plant explant, to produce a recombinant plant (for example, a recombinant plant cell comprising the nucleic acid construct) as well as to progeny thereof, and to in vitro systems that mimic biochemical or cellular components or processes in a cell.

“Cell-enhanced” and “tissue-enhanced” regulation refer to the control of gene or protein expression, for example, by a promoter, which drives expression that is not necessarily totally restricted to a single type of cell or tissue, but where expression is elevated in particular cells or tissues to a greater extent than in other cells or tissues within the organism.

A “condition-enhanced” promoter refers to a promoter that activates a gene in response to a particular environmental stimulus, for example, an abiotic stress, infection caused by a pathogen, light treatment, etc., and that drives expression in a unique pattern which may include expression in specific cell and/or tissue types within the organism (as opposed to a constitutive expression pattern in all cell types of an organism at all times).

### DESCRIPTION OF THE SPECIFIC EMBODIMENTS

The data presented herein represent the results obtained in experiments with polynucleotides that may be transformed in plants for the purpose of enhancing polysome mediated cell-type-, tissue type- or condition-enhanced RNA profiling.

The instant invention is an improvement to an existing process (polysome immunoprecipitation (IP)), whereby a cell-type specific promoter is used to drive the expression of a LexA DNA binding protein fused with the GAL4 activation protein domain which then acts in trans on two other vector components containing synthetic operator LexA (opLexA) promoter sequences fused with: a) an epitope-tagged ribosomal protein which is required to perform the polysome IP, and b) a reporter protein that is used to validate the expression pattern of the system. These components are assembled in a single vector, which also contains the required components for transformation into the desired organism (e.g., a standard plant binary vector). Alternatively, a well characterized promoter-reporter transgenic plant line (e.g., promoter::LexA-GAL4; opLexA::GFP) could be supertransformed with a second construct containing the opLexA::ribotag sequence.

The ability to characterize transformants and validate the expression patterns specified by cloned promoter sequences is critical for the implementation of any technique relying on DNA promoters. For instance, if one were creating transgenic plants that expressed such a reporter construct as described above, it would be potentially necessary to screen through dozens of transformants in order to find one with the expected expression pattern. Without the ability to use a co-regulated reporter, such as GFP, the experimentalist has to apply a method to determine if the promoter's ability to specify an expression pattern had been hindered by the location of genomic insertion; such methods are typically laborious and would likely include time and labor intensive in situ hybridization procedures. Using a co-regulated reporter, transformants can simply be visually scanned using a fluorescence microscope or other method to visualize the reporter, to select a reliable line that can be used to perform pull downs of RNA expressed in particular cells, tissues, or conditions. Importantly, once such a reliable line has been established, it can be introduced into other genetic backgrounds by techniques such as crossing or transformation, so as to enable the experimenter to examine the RNA profiles that are present in particular cells, tissues or conditions in a given transgenic or mutant genotype.

### Sequence Variations

It will readily be appreciated by those of skill in the art, that the invention includes any of a variety of polynucleotide sequences provided in the Sequence Listing or capable of encoding polypeptides that function similarly to those provided in the Sequence Listing. Due to the degeneracy of the genetic code, many different polynucleotides can encode identical and/or substantially similar polypeptides in addition to those sequences illustrated in the Sequence Listing. Nucleic acids having a sequence that differs from the sequences shown in the Sequence Listing, or complementary sequences, that encode functionally equivalent peptides (that is, peptides having some degree of equivalent or similar biological activity) but differ in sequence from the sequence shown in the sequence listing due to degeneracy in the genetic code, are also within the scope of the invention.

Altered polynucleotide sequences encoding polypeptides include those sequences with deletions, insertions, or substitutions of different nucleotides, resulting in a polynucleotide encoding a polypeptide with at least one functional characteristic of the instant polypeptides. Included within this definition are polymorphisms which may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides.

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, for example, site-directed mutagenesis, available in the art. Accordingly, any and all such variations of a sequence selected from the above table are a feature of the invention.

In addition to silent variations, other conservative variations that alter one, or a few amino acids in the encoded polypeptide, can be made without altering the function of the polypeptide. For example, substitutions, deletions and insertions introduced into the sequences provided in the Sequence Listing are also envisioned. Such sequence modifications can be engineered into a sequence by site-directed mutagenesis (for example, Olson et al., Smith et al., Zhao et al., and other articles in Wu (ed.) Meth. Enzymol. (1993) vol. 217, Academic Press) or the other methods known in the art or noted herein. Amino acid substitutions are typically of single residues; insertions usually will be on the order of about from 1 to 10 amino acid residues; and deletions will range about from 1 to 30 residues. In preferred embodiments, deletions or insertions are made in adjacent pairs, for example, a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 1 when it is desired to maintain the activity of the protein. Table 1 shows amino acids which can be substituted for an amino acid in a protein and which are typically regarded as conservative substitutions.

TABLE 1
Possible conservative amino acid substitutions

| Amino Acid Residue | Conservative substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

The polypeptides provided in the Sequence Listing have a novel activity, such as, for example, regulatory activity. Although all conservative amino acid substitutions (for example, one basic amino acid substituted for another basic amino acid) in a polypeptide will not necessarily result in the polypeptide retaining its activity, it is expected that many of these conservative mutations would result in the polypeptide retaining its activity. Most mutations, conservative or non-conservative, made to a protein but outside of a conserved domain required for function and protein activity will not affect the activity of the protein to any great extent.

### Identifying Polynucleotides or Polypeptides Related to the Disclosed Sequences by Percent Identity

With the aid of a computer, one of skill in the art could identify all of the polypeptides, or all of the nucleic acids that encode a polypeptide, with, for example, at least 85% identity to the sequences provided herein and in the Sequence Listing. Electronic analysis of sequences may be conducted with a software program such as the MEGALIGN program (DNASTAR, Inc. Madison, Wis.). The MEGALIGN program can create alignments between two or more sequences according to different methods, for example, the clustal method (see, for example, Higgins and Sharp (1988) Gene 73: 237-244). The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, for example, each amino acid gap is weighted as if it were a single amino acid or nucleotide mismatch between the two sequences (see U.S. Pat. No. 6,262,333).

Software for performing BLAST analyses is publicly available, for example, through the National Center for Biotechnology Information (see internet website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, 1990, supra; Altschul et al., 1993, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff (1989) Proc. Natl. Acad. Sci. USA 89: 10915). Unless otherwise indicated for comparisons of predicted polynucleotides, “sequence identity” refers to the % sequence identity generated from a tblastx using the NCBI version of the algorithm at the default settings using gapped alignments with the filter “off” (see, for example, internet website at www.ncbi.nlm.nih.gov/).

Other techniques for alignment are described by Doolittle, ed. (1996) Methods in Enzymology, vol. 266: “Computer Methods for Macromolecular Sequence Analysis” Academic Press, Inc., San Diego, Calif., USA. Preferably, an alignment program that permits gaps in the sequence is utilized to align the sequences. The Smith-Waterman is one type of algorithm that permits gaps in sequence alignments (see Shpaer (1997) Methods Mol. Biol. 70: 173-187). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Percent identity can also be determined manually, by comparing the entire length of a sequence of sequence with another in an optimal alignment.

Generally, the percentage similarity between two polypeptide sequences, for example, sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, for example, the Jotun Hein method (see, for example, Hein (1990)Methods Enzymol. 183: 626-645) Identity between sequences can also be determined by other methods known in the art, for example, by varying hybridization conditions (see US Patent Application No. US20010010913).

At the polynucleotide level, the sequences described herein in the Sequence Listing, and the sequences of the invention by virtue of a paralogous or homologous relationship with the sequences described in the Sequence Listing, will typically share at least about 30%, or 40% nucleotide sequence identity, preferably at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to one or more of the listed full-length sequences, or to a region of a listed sequence excluding or outside of the region(s) encoding a known consensus sequence or consensus DNA-binding site, or outside of the region(s) encoding one or all conserved domains. The degeneracy of the genetic code enables major variations in the nucleotide sequence of a polynucleotide while maintaining the amino acid sequence of the encoded protein.

At the polypeptide level, the sequences described herein in the Sequence Listing and Table 1, and the sequences of the invention by virtue of a paralogous or homologous relationship with the sequences described in the Sequence Listing or in Table 1, will typically share at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% amino acid sequence identity or more sequence identity to one or more of the listed full-length sequences, or to a listed sequence but excluding or outside of the known consensus sequence or consensus DNA-binding site.

### Identifying Polynucleotides Related to the Disclosed Sequences by Hybridization

Polynucleotides homologous to the sequences illustrated in the Sequence Listing and tables can be identified, for example, by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited below (for example, Sambrook et al. (1989) Molecular Cloning: A Laboratory Manual, 2nd Ed., Cold Spring Harbor Laboratory, Cold Spring Harbor, N.Y.; Schroeder et al. (2002) Current Biol. 12, 1462-1472; Berger and Kimmel (1987), “Guide to Molecular Cloning Techniques”, in Methods in Enzymology, vol. 152, Academic Press, Inc., San Diego, Calif.; and Anderson and Young (1985) “Quantitative Filter Hybridisation”, In: Hames and Higgins, ed., Nucleic Acid Hybridisation, A Practical Approach. Oxford, IRL Press, 73-111).

Encompassed by the invention are polynucleotide sequences that are capable of hybridizing to the claimed polynucleotide sequences, including any of the polynucleotides within the Sequence Listing, and fragments thereof under various conditions of stringency (see, for example, Wahl and Berger (1987) Methods Enzymol. 152: 399-407; and Kimmel (1987) Methods Enzymol. 152: 507-511). In addition to the nucleotide sequences listed in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

With regard to hybridization, conditions that are highly stringent, and means for achieving them, are well known in the art. See, for example, Sambrook et al., 1989; Berger, 1987, pages 467-469; and Anderson and Young, 1985, all supra.

Stability of DNA duplexes is affected by such factors as base composition, length, and degree of base pair mismatch. Hybridization conditions may be adjusted to allow DNAs of different sequence relatedness to hybridize. The melting temperature (Tₘ) is defined as the temperature when 50% of the duplex molecules have dissociated into their constituent single strands. The melting temperature of a perfectly matched duplex, where the hybridization buffer contains formamide as a denaturing agent, may be estimated by the following equations:

Tₘ(° C.)=81.5+16.6(log [Na+])+0.41(% G+C)−0.62(% formamide)−500/L (I) DNA-DNA

Tₘ(° C.)=79.8+18.5(log [Na+])+0.58(% G+C)+0.12(% G+C)²−0.5(% formamide)−820/L (II) DNA-RNA

Tₘ(° C.)=79.8+18.5(log [Na+])+0.58(% G+C)+0.12(% G+C)²−0.35(% formamide)−820/L (III) RNA-RNA

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson and Young, 1985, supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tₘ−5° C. to Tₘ−20° C., moderate stringency at Tₘ−20° C. to Tₘ−35° C. and low stringency at Tₘ−35° C. to Tₘ−50° C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at Tₘ−25° C. for DNA-DNA duplex and Tₘ−15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or Northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, for example, to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, for example, formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, for example, sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present polypeptides include, for example:

6×SSC and 1% SDS at 65° C.;

50% formamide, 4×SSC at 42° C.; or

0.5×SSC to 2.0×SSC, 0.1% SDS at 50° C. to 65° C.;

with a first wash step of, for example, 10 minutes at about 42° C. with about 20% (v/v) formamide in 0.1×SSC, and with, for example, a subsequent wash step with 0.2×SSC and 0.1% SDS at 65° C. for 10, 20 or 30 minutes. An example of an amino acid sequence of the invention would include one encoded by a polynucleotide selected from the Sequence Listing and nucleic acid sequence fragments encoding various proteins that have been or can be used for cloning and nucleic acid sequence fragments that encode various functional (e.g., regulatory or indicator) polypeptides, and which can be incorporated into nucleic acid constructs for cloning purposes.

Useful variations on these conditions will be readily apparent to those skilled in the art.

A person of skill in the art would not expect substantial variation among polynucleotide species encompassed within the scope of the present invention because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 minutes, or about 0.1×SSC, 0.1% SDS at 65° C. and washing twice for 30 minutes. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, for example, 50° C.

An example of a low stringency wash step employs a solution and conditions of at least 25° C. in 30 mM NaCl, 3 mM trisodium citrate, and 0.1% SDS over 30 minutes. Greater stringency may be obtained at 42° C. in 15 mM NaCl, with 1.5 mM trisodium citrate, and 0.1% SDS over 30 minutes. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (see, for example, US Patent Application No. US20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a polypeptide known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, for example, a fluorescent label, a colorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the invention are polynucleotide sequences that are capable of hybridizing to the claimed polynucleotide sequences, including any of the polynucleotides within the Sequence Listing, and fragments thereof under various conditions of stringency (see, for example, Wahl and Berger, 1987, pages 399-407; and Kimmel, 1987). In addition to the nucleotide sequences in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

### EXAMPLES

It is to be understood that this invention is not limited to the particular devices, machines, materials and methods described. Although particular embodiments are described, equivalent embodiments may be used to practice the invention.

The invention, now being generally described, will be more readily understood by reference to the following examples, which are included merely for purposes of illustration of certain aspects and embodiments of the present invention and are not intended to limit the invention. It will be recognized by one of skill in the art that a polypeptide that is associated with a particular first trait may also be associated with at least one other, unrelated and inherent second trait which was not predicted by the first trait.

### Example I

### Generation of Constructs and Cloning Information

Plants containing a “multi-component 3-way” RiboTag vector (e.g., SEQ ID NO: 1) were generated. The correct and desired expression pattern was identified for multiple promoters, including the constitutive 35S (SEQ ID NO: 17), green tissue RBCS1A (SEQ ID NO: 10), vascular SUC2 (SEQ ID NO: 9), and stomate G682 (SEQ ID NO: 18). Other cell-type specific promoters could also be used, such as the meristematic STM1 (SEQ ID NO: 11), WUSCHEL (SEQ ID NO: 20), and CLAVATA3 (SEQ ID NO: 21), root specific SCR1 (SEQ ID NO: 12), root specific SHR1 (SEQ ID NO: 13), dividing tissue CYCD3 (SEQ ID NO: 14), floral meristem AP1 (SEQ ID NO: 15), APETALA3 (SEQ ID NO 22), PISTILLATA (SEQ ID NO 23), epidermal CUT1 (SEQ ID NO: 16), or a variety of other promoters driving desired expression patterns (such as in a cell-enhanced, tissue-enhanced, or condition-enhanced expression pattern).

Also of interest in the present invention is light-mediated regulation of gene or protein expression, such as can be mediated by a promoter that acts in light signal transduction such as the G1988 promoter (SEQ ID NO: 19), the ELIP promoter (SEQ ID NO 25), the HY5 promoter (SEQ ID NO 26), or the SPA1 promoter (SEQ ID NO 24). The method herein would allow the experimentalist to examine the RNA populations present in the particular cells or tissues within the plant that are responding to a light treatment. Similar studies could be done to examine the RNA populations present in the particular cells or tissues within the plant that are responding to an environmental stress such as drought, nutrient limitation or pathogen attack. Examples of promoters that could be used to examine RNA in drought responding cell include SEQ ID NOs: 66-74, and in pathogen responding cells include SEQ ID NOs: 27-65.

Indeed, the method described in the present application can be applied with any promoter that drives a distinct and recognizable pattern, to examine the RNA populations that are present and associated with ribosomes within the same cells in which that promoter drives expression.

### Example II

### Transformation of Agrobacterium with the Expression Vector

After the expression constructs were generated, the constructs were used to transform Agrobacterium tumefaciens cells expressing the gene products. The stock of Agrobacterium tumefaciens cells for transformation were made as described by Nagel et al. (1990) FEMS Microbiol Letts. 67: 325-328. Agrobacterium strain ABI was grown in 250 ml LB medium (Sigma) overnight at 28° C. with shaking until an absorbance over 1 cm at 600 nm (A₆₀₀) of 0.5-1.0 was reached. Cells were harvested by centrifugation at 4,000×g for 15 min at 4° C. Cells were then resuspended in 250 μl chilled buffer (1 mM HEPES, pH adjusted to 7.0 with KOH). Cells were centrifuged again as described above and resuspended in 125 μl chilled buffer. Cells were then centrifuged and resuspended two more times in the same HEPES buffer as described above at a volume of 100 μl and 750 μl, respectively. Resuspended cells were then distributed into 40 μl aliquots, quickly frozen in liquid nitrogen, and stored at −80° C.

Agrobacterium cells were transformed with constructs prepared as described above following the protocol described by Nagel et al. (supra). For each DNA construct to be transformed, 50-100 ng DNA (generally resuspended in 10 mM Tris-HCl, 1 mM EDTA, pH 8.0) was mixed with 40 μl of Agrobacterium cells. The DNA/cell mixture was then transferred to a chilled cuvette with a 2 mm electrode gap and subject to a 2.5 kV charge dissipated at 25 μF and 200 μF using a Gene Pulser II apparatus (Bio-Rad, Hercules, Calif.). After electroporation, cells were immediately resuspended in 1.0 ml LB and allowed to recover without antibiotic selection for 2-4 hours at 28° C. in a shaking incubator. After recovery, cells were plated onto selective medium of LB broth containing 100 μg/ml spectinomycin (Sigma) and incubated for 24-48 hours at 28° C. Single colonies were then picked and inoculated in fresh medium. The presence of the plasmid construct was verified by PCR amplification and sequence analysis.

### Example III

### Transformation of Plants with Agrobacterium tumefaciens

After transformation of Agrobacterium tumefaciens with the constructs or plasmid vectors containing the gene of interest, single Agrobacterium colonies were identified, propagated, and used to transform plants. In the example here, we detail transformation of Arabidopsis plants, but the constructs could be introduced into any plant species, including crops such as corn, soybean, cotton, rice, canola and tomato, which are amenable to transformation. Briefly, 500 ml cultures of LB medium containing 50 mg/l kanamycin were inoculated with the colonies and grown at 28° C. with shaking for 2 days until an optical absorbance at 600 nm wavelength over 1 cm (A₆₀₀) of >2.0 is reached. Cells were then harvested by centrifugation at 4,000×g for 10 min, and resuspended in infiltration medium (½× Murashige and Skoog salts (Sigma), 1× Gamborg's B-5 vitamins (Sigma), 5.0% (w/v) sucrose (Sigma), 0.044 μM benzylamino purine (Sigma), 200 μl/l Silwet L-77 (Lehle Seeds) until an A₆₀₀ of 0.8 was reached.

Prior to transformation, Arabidopsis thaliana seeds (ecotype Columbia) were sown at a density of ˜10 plants per 4″ pot onto Pro-Mix BX potting medium (Hummert International) covered with fiberglass mesh (18 mm×16 mm). Plants were grown under continuous illumination (50-75 μE/m²/sec) at 22-23° C. with 65-70% relative humidity. After about 4 weeks, primary inflorescence stems (bolts) are cut off to encourage growth of multiple secondary bolts. After flowering of the mature secondary bolts, plants were prepared for transformation by removal of all siliques and opened flowers.

The pots were then immersed upside down in the mixture of Agrobacterium infiltration medium as described above for 30 sec, and placed on their sides to allow draining into a 1′×2′ flat surface covered with plastic wrap. After 24 h, the plastic wrap was removed and pots are turned upright. The immersion procedure was repeated one week later, for a total of two immersions per pot. Seeds were then collected from each transformation pot and analyzed following the protocol described below. Other standard methods of plant transformation, such as particle bombardment, or tissue culture-based Agrobacterium cocultivation could also be applied to transform Arabidopsis, or any other plant species of interest.

### Example IV

### Identification of Arabidopsis Primary Transformants

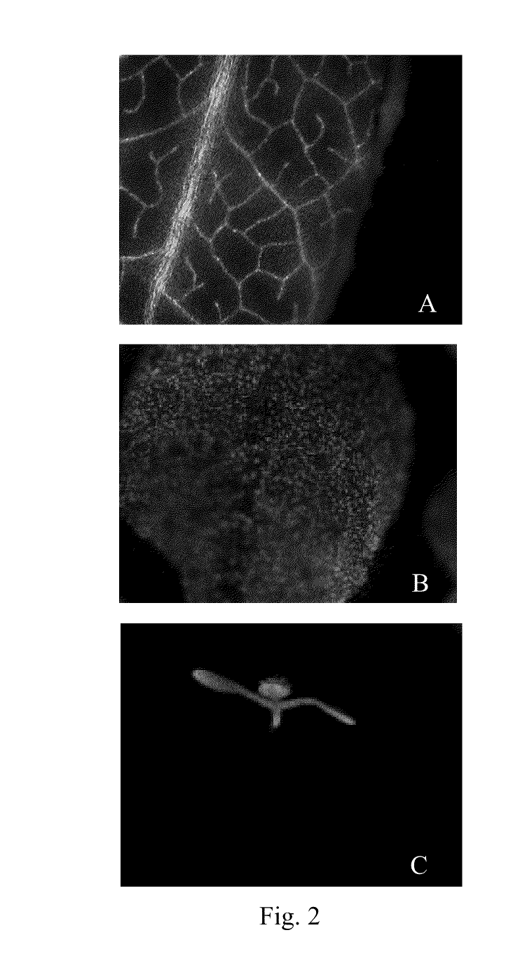

Seeds collected from the transformation pots were sterilized essentially as follows. Seeds were dispersed into in a solution containing 0.1% (v/v) Triton X-100 (Sigma) and sterile water and washed by shaking the suspension for 20 min. The wash solution was then drained and replaced with fresh wash solution to wash the seeds for 20 min with shaking. After removal of the ethanol/detergent solution, a solution containing 0.1% (v/v) Triton X-100 and 30% (v/v) bleach (CLOROX; Clorox Corp. Oakland Calif.) was added to the seeds, and the suspension was shaken for 10 min. After removal of the bleach/detergent solution, seeds were then washed five times in sterile distilled water. The seeds were stored in the last wash water at 4° C. for 2 days in the dark before being plated onto antibiotic selection medium (1× Murashige and Skoog salts (pH adjusted to 5.7 with 1M KOH), 1× Gamborg's B-5 vitamins, 0.9% phytagar (Life Technologies), and 50 mg/l kanamycin). Seeds were germinated under continuous illumination (50-75 μE/m²/sec) at 22-23° C. After 7-10 days of growth under these conditions, kanamycin resistant primary transformants (T1 generation) were visible and obtained. At this stage, transformed plants were subjected to detailed microscopic analysis to verify that each cloned promoter fragment was driving gene expression in the desired cell type-specific pattern. Currently, this technology has been applied to validate transgenic Arabidopsis lines for multiple promoters, and this work will be ongoing for additional promoters. While still growing on primary selection plates, seedlings were placed under a fluorescent dissecting microscope so that the opLexA::GFP protein pattern could be verified. This pattern, since it was controlled via a GAL4-LexA 2-component system, also represented the pattern of the opLexA::RiboTag. Plants showing the correct SUC2 promoter pattern, for example, showed high levels of fluorescence in the vascular tissue of the leaves and roots. Plants containing the correct RBCS1A promoter (SEQ ID NO: 10) pattern showed strong expression in green tissue, but not in roots. The G682 promoter (SEQ ID NO: 18) showed enhanced expression in guard cells. Representative images of these patterns are shown in FIG. 2. Seedlings showing the desired expression pattern were then transplanted to soil (Pro-Mix BX potting medium) for continued growth and characterization at subsequent developmental stages.

Primary transformants were self fertilized and progeny seeds (T₂) collected; kanamycin resistant seedlings were selected and analyzed. The expression levels of the recombinant polynucleotides in the transformants typically varies from about a 5% expression level increase to at least a 100% expression level increase, in tissue samples from the transgenic lines compared to those from wild-type controls. Similar observations are made with respect to polypeptide level expression.

### Example IV

### Polysome Immunoprecipitation and Transcript Profiling

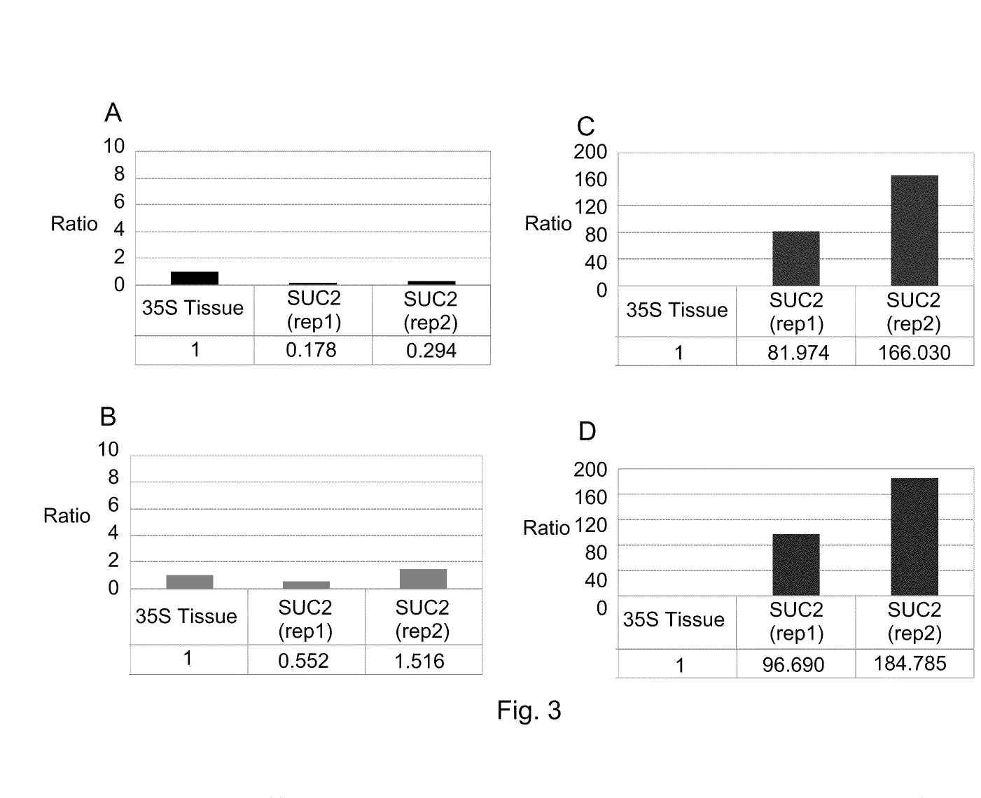

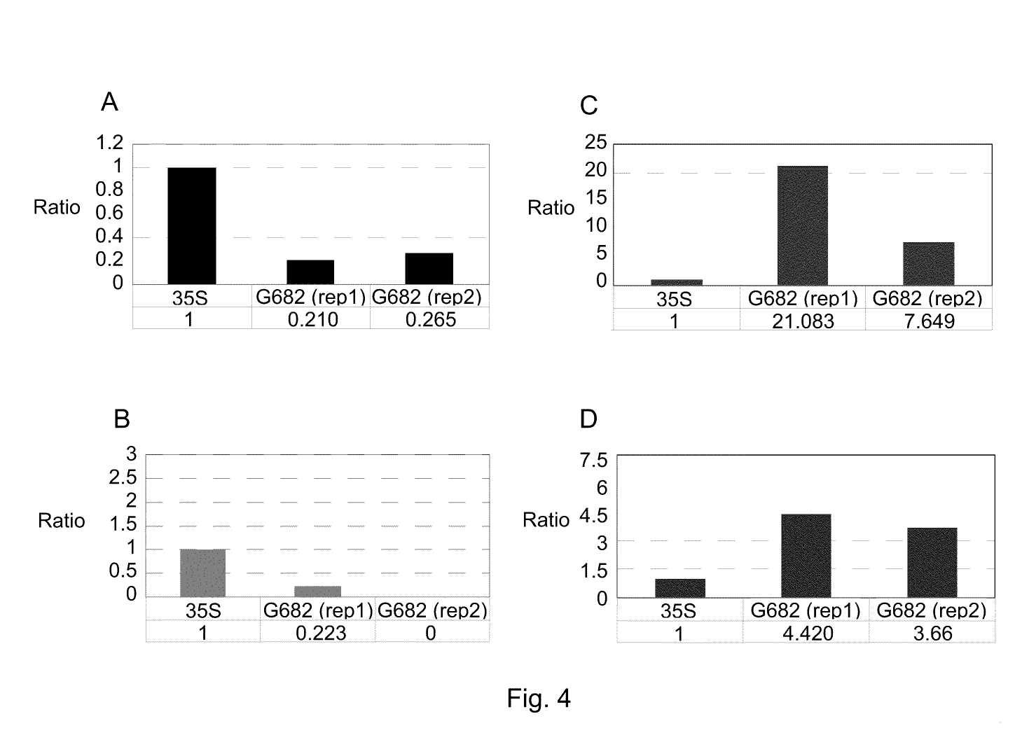

Validated transgenic plant lines have subsequently been used in polysome immunoprecipitations according to the protocol modified from Zanetti et al., 2005. Briefly, frozen tissue is pulverized in liquid nitrogen, homogenized in polysome extraction buffer, and a polysome-containing supernatant is immunopreciptated as per the protocol. Once polysomes are isolated, RNA is extracted and prepared for analysis by, e.g., qPCR, microarray transcript profiling, high throughput sequencing or other methods. Enrichment of polysome-associated mRNA transcripts from specific cell types was verified by performing qPCR on markers of that cell type. For example, specific enrichment of SUC2 (SEQ ID NO: 8) and AHA3 (AT5G57350, SEQ ID NO: 6) mRNA was verified in prSUC2::RiboTag plants by a factor of greater than 100-fold. Similarly, guard cell specific transcripts could be specifically enriched in pull-downs from prG682::RiboTag plants. These results are shown in FIGS. 3 and 4, respectively. Following such verification, these isolated RNA populations are prepared for expression profiling using Affymetrix microarrays, or other such expression analysis systems (e.g., cDNA microarrays, high throughput (e.g., Solexa/Illumina) sequencing, etc.).

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the appended claims. Modifications that become apparent from the foregoing description and accompanying figures fall within the scope of the claims.

What is claimed is:

1. A transgenic plant transformed with at least one nucleic acid construct, wherein the at least one nucleic acid construct comprises, in 5′ to 3′ order:
(a) a first nucleic acid sequence comprising a promoter that is fused to a subsequence encoding a transcriptional activator/DNA binding polypeptide sequence, wherein expression of the subsequence is regulated by the promoter;
(b) a second nucleic acid sequence comprising a binding site for the transcriptional activator/DNA binding polypeptide sequence of (a) fused to a sequence encoding a transcriptional activator target sequence comprising a DNA target site fused to a ribosomal protein coding sequence with a protein epitope coding region, wherein the transcriptional activator/DNA binding polypeptide sequence of (a) may bind to the transcriptional activator target sequence;
(c) a third nucleic acid sequence comprising a binding site for the transcriptional activator in (a) fused to a sequence encoding a reporter protein; and
(d) optionally, a fourth nucleic acid sequence encoding an antibiotic resistance marker.

2. The transgenic plant of claim 1, wherein the promoter is a cell type-, tissue type-, or condition-enhanced promoter.

3. The transgenic plant of claim 1, wherein the promoter is selected from the group consisting of SEQ ID NOs: 9-74.

4. The transgenic plant of claim 1, wherein the transcriptional activator/DNA binding polypeptide sequence is a polypeptide having a LexA DNA binding domain fused to a GAL4 activation domain.

5. The transgenic plant of claim 4, wherein the transcriptional activator target sequence is an opLexA sequence to which the LexA DNA binding domain-GAL4 activation domain fusion polypeptide binds and activates transcription.

6. The transgenic plant of claim 1, wherein the second nucleic acid sequence encodes a transcriptional activator target sequence::HIS-FLAG-Ribosomal protein fusion.

7. The transgenic plant of claim 1, wherein the reporter protein is selected from the group consisting or green fluorescent protein, yellow fluorescent protein, red fluorescent protein, beta-glucuronidase, and luciferase.

8. The transgenic plant of claim 1, wherein the ribosomal protein coding sequence encodes RPL18_ARATH 60S ribosomal protein L18 (SEQ ID NO: 2).

9. The transgenic plant of claim 1, wherein the ribosomal protein coding sequence comprises SEQ ID NO: 3.

10. The transgenic plant of claim 1, wherein the at least one nucleic acid construct is introduced into the transgenic plant by crossing or transforming the transgenic plant.

11. The transgenic plant of claim 1, wherein the transgenic plant is a host plant cell.

12. A method for improving polysome-mediated RNA profiling, said method comprising the steps of:
transforming a plant by introducing into the plant at least one nucleic acid construct, wherein the at least one nucleic acid construct comprises, in 5′ to 3′ order:
(a) a first nucleic acid sequence comprising a promoter that is fused to a subsequence encoding a transcriptional activator/DNA binding polypeptide sequence, wherein expression of the subsequence is regulated by the promoter;
(b) a second nucleic acid sequence comprising a binding site for the transcriptional activator/DNA binding polypeptide sequence of (a) fused to a sequence encoding a transcriptional activator target sequence comprising a DNA target site fused to a ribosomal protein coding sequence with a protein epitope coding region, wherein the transcriptional activator/DNA binding polypeptide sequence of (a) may bind to the transcriptional activator target sequence;
(c) a third nucleic acid sequence comprising a binding site for the transcriptional activator in (a) fused to a sequence encoding a reporter protein; and
(d) optionally, a fourth nucleic acid sequence encoding an antibiotic resistance marker.

13. The method of claim 12, wherein the promoter is a cell type-, tissue type-, or condition-enhanced promoter.

14. The method of claim 12, wherein the promoter is selected from the group consisting of SEQ ID NOs: 9-74.

15. The method of claim 12, wherein the transcriptional activator/DNA binding polypeptide sequence is a polypeptide having a LexA DNA binding domain fused to a GAL4 activation domain.

16. The method of claim 15, wherein the transcriptional activator target sequence is an opLexA sequence to which the LexA DNA binding domain-GAL4 activation domain fusion polypeptide binds and activates transcription.

17. The method of claim 12, wherein the second nucleic acid sequence encodes a transcriptional activator target sequence::HIS-FLAG-Ribosomal protein fusion.

18. The method of claim 12, wherein the reporter protein is selected from the group consisting or green fluorescent protein, yellow fluorescent protein, red fluorescent protein, beta-glucuronidase, and luciferase.

19. The method of claim 12, wherein the ribosomal protein coding sequence encodes RPL18_ARATH 60S ribosomal protein L18 (SEQ ID NO: 2).

20. The method of claim 12, wherein the ribosomal protein coding sequence comprises SEQ ID NO: 3.

21. A nucleic acid construct comprising, in 5′ to 3′ order:
(a) a first nucleic acid sequence comprising a promoter that is fused to a subsequence encoding a transcriptional activator/DNA binding polypeptide sequence, wherein expression of the subsequence is regulated by the promoter;
(b) a second nucleic acid sequence comprising a binding site for the transcriptional activator/DNA binding polypeptide sequence of (a) fused to a sequence encoding a transcriptional activator target sequence comprising a DNA target site fused to a ribosomal protein coding sequence with a protein epitope coding region, wherein the transcriptional activator/DNA binding polypeptide sequence of (a) may bind to the transcriptional activator target sequence;
(c) a third nucleic acid sequence comprising a binding site for the transcriptional activator in (a) fused to a sequence encoding a reporter protein; and
(d) optionally, a fourth nucleic acid sequence encoding an antibiotic resistance marker.


SEQUENCE LISTING (74 sequences)

SEQ ID NO: 1 - DNA, 7629 bp, artificial sequence
  ribotag vector P28871 MCS (355-387), LexA- GAL4 (519-1475), E9-term (1476-2145), 6xHIS-FLAG RPL18 (2482- 3123), OCS-term (3125-3835), GFP (4221-4955), E9-term (4956-5635), Kanamycin resistance (6013-6807)
ctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccccatcaagcttggtacccgggtcgacaaactagtaagcggccgcaagacccttcctctatataaggaagttcatttcatttggagaggacacgctcgagtataagagctcatttttacaacaattaccaacaacaacaaacaacaaacaacattacaattacatttacaattaccatggaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtctgttgcaagaagaggaagaagggttgccgctggtaggtcgtgtggctgccggtgaaccacttctggcgcaacagcatattgaaggtcattatcaggtcgatccttccttattcaagccgaatgctgatttcctgctgcgcgtcagcgggatgtcgatgaaagatatcggcattatggatggtgacttgctggcagtgcataaaactcaggatgtacgtaacggtcaggtcgttgtcgcacgtattgatgacgaggttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattccccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatctagtacatatccatatctaatcttacctcgactgctgtatataaaaccagtggttatatgtccagtactgctgtatataaaaccagtggttatatgtacagtacgtcgatcgactgctgtatataaaaccagtggttatatgtacagtactgctgtatataaaaccagtggttatatgtacagtacgtcgaggggatgatcaagacccttcctctatataaggaagttcatttcatttggagaggacacgctgacaagctgactctagctctagtctagagtataagagctctatttttacaacaattaccaacaacaacaaacaacaaacaacattacaattacatttacaattaccatgggacatcaccatcatcaccatggtggaggtgattataaggatgatgatgataagggaggtggtggaggaggtggatccggtattgatctgatcgccggaggtaagagtaagaagaccaaaaggacagctccaaagtctgatgatgtctacctcaagcttactgtcaagctttaccggtttctggtaaggagaactaatagcaagttcaatggtgtgatattgaagaggcttttcatgagcaaagtcaacaaagctcctctttctctatctaggcttgtggagttcatgactggcaaggaagataagattgccgtcttggttggaactataactgatgatttgagggtacacgagattccagccatgaaagtgactgccttgaggttcacagagagagcaagggctcgcattgagaaagctggaggtgaatgcttaacctttgaccagctcgctctcagagctccattgggccagaacacggttcttcttagaggacctaagaattcacgtgaagcagtgaagcatttcggacctgctcctggtgtgccacacagtcactccaagccatatgttcgggccaagggaaggaagttcgagaaggccagaggaaagaggaagagtcgtggattcaaggtttaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactagaacatatccatatctaatcttacctcgactgctgtatataaaaccagtggttatatgtccagtactgctgtatataaaaccagtggttatatgtacagtacgtcgactgctgtatataaaaccagtggttatatgtacagtactgctgtatataaaaccagtggttatatgtacagtacgtcgaggggatgatcaagacccttcctctatataaggaagttcatttcatttggagaggacacgctcgagtataagagctcatttttacaacaattaccaacaacaacaaacaacaaacaacattacaattacatttacaattaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagccactcgaagcggccggccgccactcgagtggaagctagctttccgatcctacctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatacttccactgacgtaagggatgacgcacaatcccactatccttcgcaagacccttcctctatataaggaagttcatttcatttggagaggacacgctgaaatcaccagtctctctctacaagatcggggatctctagctagacgatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgatccccaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatcgggccactcgagtggtggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttcctagcttggctgccatttttggggtgaggccgttcgcggccgaggggcgc

SEQ ID NO: 2 - protein, 187 aa, Arabidopsis thaliana
  RL18_ARATH 60S ribosomal protein L18
MGIDLIAGGKSKKTKRTAPKSDDVYLKLTVKLYRFLVRRTNSKFNGVILKRLFMSKVNKAPLSLSRLVEFMTGKEDKIAVLVGTITDDLRVHEIPAMKVTALRFTERARARIEKAGGECLTFDQLALRAPLGQNTVLLRGPKNSREAVKHFGPAPGVPHSHSKPYVRAKGRKFEKARGKRKSRGFKV

SEQ ID NO: 3 - DNA, 564 bp, Arabidopsis thaliana
  coding sequence for RL18_ARATH 60S ribosomal protein L18
atgggtattgatctgatcgccggaggtaagagtaagaagaccaaaaggacagctccaaagtctgatgatgtctacctcaagcttactgtcaagctttaccggtttctggtaaggagaactaatagcaagttcaatggtgtgatattgaagaggcttttcatgagcaaagtcaacagagctcctctttctctatctaggcttgtggagttcatgactggcaaggaagataagattgccgtcttggttggaactataactgatgatttgagggtacacgagattccagccatgaaagtgactgccttgaggttcacagagagagcaagggctcgcattgagaaagctggaggtgaatgcttaacctttgaccagctcgctctcagagctccattgggccagaacacggttcttcttagaggacctaagaattcacgtgaagcagtgaagcatttcggacctgctcctggtgtgccacacagtcactccaagccatatgttcgggccaagggaaggaagttcgagaaggccagaggaaagaggaagagtcgtggattcaaggtttaa

SEQ ID NO: 4 - DNA, 789 bp, Arabidopsis thaliana
  AT2G01520, MLP328
attcatcaagtaaagaaacaaacaaacaaacaaaaaacacacttcactcgctacacaaggaagagagatggcgacgtcaggaacatatgtgacggaggttccattgaaaggatcggctgagaaacactacaagcggtggaggagtgagaaccacctcttccccgacgccatcggccaccacatccaaggtgtcaccatccacgacggtgaatgggactcccatggagccatcaagatttggaactacacatgcgatgggaaaccggaggtgttcaaggagaggagagagatagacgatgagaatatggcggtaacgttcagaggactcgaaggtcacgtgatggagcagcttaaagtgtatgacgtcatctttcagttcattcaaaagtcacctgatgatatcatctgtaagatcactatgatctgggagaagcaaaacgatgacatgcctgagcccagcaactacatgaagttcgtcaagagcctcgctgctgacatggatgatcacgttctcaaagcctaaatatccctttcacatcaacccatcatcacctaatatcatgatcatcatcaccctcatacgtacattgctccttttgtgtgtgtgtgtgtgtatcgtttttatggtgttttcaatataataaatgggggtttacatgtatggtttcatctactataatatgctttgatgttgtttattatggttgaggtttcatgtatgctcatctacgcatgtatccttgttaattaatgattttcaagagttatcaatcaatactgccctttttt

SEQ ID NO: 5 - DNA, 554 bp, Arabidopsis thaliana
  AT1G22690, GC1 gibberellin-responsive protein
acactacttcaaaatcactactcaagaaatatgaagaagatgaatgtggtggcttttgttacgctgatcatctcttttcttctgctttctcaggtacttgcagagttgtcatcatccagcaacaatgaaacttcctctgtttctcagacgaatgacgagaaccaaactgcggcgtttaagagaacataccaccatcgtccaagaatcaattgtgggcatgcatgcgcaaggagatgcagtaagacatcgaggaagaaagtttgtcacagagcctgtggaagttgttgtgccaagtgtcagtgtgtgccgccgggaacctccggcaacacagcatcatgtccttgctacgccagtatccgtacacatggcaataaactcaaatgtccttaaaagacttctcatttctcaactatagtctcatcttctgattatgtttcttcttttgttatgttgcatgtgtgatgtgtgagcttattattatgttgattgttgacataattcaactatataatttgtatcgattccgaataataagatgagtgattttattggct

SEQ ID NO: 6 - DNA, 3226 bp, Arabidopsis thaliana
  AT5G57350, AHA3
accttcacttgctctcttttttccgaagaatctggcacagagagaagatggcgagtggcctcgaggatatcgtgaacgagaatgttgatttggagaaaatacctattgaggaagtgttccagcagttgaaatgcagcagagaaggtttatctggagcagaaggagaaaacagactccagatctttggccccaacaaactcgaggagaagaaggaaagcaagttactcaagttcttgggttttatgtggaaccctctctcttgggtcatggaagcagctgcaatcatggctattgccttggctaatggcggtggaaagccaccggattggcaagattttgtcggtatcgtttgtcttttggttatcaattcaactatcagttttgtcgaagaaaacaatgctggaaatgctgctgctgctcttatggctggtcttgctcccaaaactaaggttctaagagatggcaaatggtcagagcaagaagcttctattcttgttcctggagatattgtgagcatcaagcttggtgacatcattcctgctgatgcacgtcttcttgaaggtgatcctttaaaagtcgaccaatctgctttgaccggagaatctcttcccgcaacgaaaggtccaggggaagaagttttctccggttctacctgcaagcaaggtgagattgaagcggttgtgatagccactggagtccatactttcttcggtaaagcggctcatctagtcgacagcacaaaccaagttggacatttccagaaagttcttacggcgattggaaacttctgcatatgttccatagctgttggtattgcgattgagatcgttgtcatgtatccgatccagcgcagacactacagagacggaatcgacaatcttcttgttcttttgattggtggtatccccattgcaatgcctactgtcttgtctgtcacaatggctattggatctcacaagttgtctcaacaaggagctatcacaaaacgaatgaccgccattgaagaaatggctggtatggatgttctgtgcagcgataaaaccgggactctcacgctaaacaagcttagtgttgacaagaatcttattgaggtttattgtaaaggcgttgagaaagatgaagttttgctttttgctgctagagcttcaagagttgagaaccaagatgctattgatgctgctatggttggaatgctcgctgatccaaaagaggcaagagctggaatcagagagattcacttcctaccattcaatccagttgataagcgaaccgctttgacgtttatcgacagcaacggaaactggcaccgagtaagcaaaggagctcccgagcagattcttgatctctgtaacgcgagagctgatctgaggaagagagtccactctacaatcgataagtacgctgagcgtggacttaggtcattagctgtttcaagacagactgtaccagagaaaacaaaagaaagctctggtagtccatgggaatttgttggtgtgttgcctctgtttgatcctccaagacatgatagtgccgaaaccattagaagagctctagacctcggtgtcaatgtcaagatgattactggtgatcaacttgctattgcgaaagagacaggacgtagacttggaatgggatcaaacatgtacccatcctcttctttactcggtaaacacaaagacgaagccatggctcacattcctgttgaggatttgattgagaaagctgatggctttgctggagtcttcccagagcacaaatacgaaattgtgaagaagttgcaagaaaggaagcatatctgtggtatgactggagatggagtgaatgacgctccagcattgaagaaagcggatataggtatcgctgtggcggatgcaactgacgctgcacgaggtgcttctgatattgtcctcaccgagccaggtctcagtgtgatcatcagcgcggttctaaccagtagagccatcttccagagaatgaagaactacacaatatatgcagtctcaattaccattcgtatagtgtttgggtttatgctcattgctctaatatggaagttcgacttttcaccattcatggttttgatcattgctatattaaacgatggaaccatcatgactatctcaaaggacagagtcaagccttctccaacaccagatagctggaaactcaaagaaatcttcgcaaccggcgttgttcttggaggctacatggctataatgactgttgttttcttctgggccgcatacaaaaccgatttcttcccgagaacattccacgtgagagacttgagaggcagtgaacatgagatgatgtctgctttatacttacaagtcagtattgtgagccaagctcttatattcgtcactcgatctagaagctggtcttttacagaacgacctggatatttcttgctaattgctttctgggtagcacaattgattgcaacggctattgcggtttacggtaattgggaatttgcaagaatcaaaggaataggatggggatgggctggagtaatctggctttacagtattgtcttctactttccattagacataatgaaattcgcaatccgttacatactagccggaacagcctggaagaatatcattgacaacaggactgcgtttaccactaagcaaaactatggaatagaggagagggaagcacaatgggctcatgcacaaaggactttacacggtcttcagaacactgaaacagccaacgttgtcccggagagaggtggttacagagaactgtctgagattgcaaatcaagccaagagacgagctgagatcgcaaggcttagggaacttcatacgcttaaaggacatgtagagtcagtggtgaagctaaagggactagacattgagacagctggtcactacaccgtttaataaagatttaacaagaagatgattaaggaagaagaagatccttttgtttaagagatgataatgatgatgattttataacaagagaataaggttttttttctgtgtatgttaagttcctctaaactcctggggtttttcttccttccttattcatggttttagactttaattataaccatgtgtttttgtttcccctctcttgaataaaaaagagaagatactaatatcataggtttaatacgtttatgttttataaaagttcaatagatattgtttttggatattatgtgaacataacaatatttataaattcacctttcaattttctt

SEQ ID NO: 7 - DNA, 2289 bp, Arabidopsis thaliana
  AT5G46240, KAT1
ataacaaatgggtcacaacaagttgccataaaggtcccaacagaagaagaaaagagaagtttccccgtgttgctttgtcacaagcttcatcactatttataactaccttgccaagaagaaagcctaaaaaggtataacagtttctttgtctttagagatcatcaaaaagatgtcgatctcttggactcgaaatttcttcgaaagattctgcgtcgaggaatacaatatagacaccataaaacagagtagtttcctctctgccgatcttctaccatctcttggagccaggatcaaccaatctactaagctccgcaaacacataatctctccttttaatccacgttacagagcgtgggagatgtggctagtattactagttatttactcagcttggatttgcccatttcaatttgctttcatcacctataaaaaagacgcgattttcatcatcgacaacattgttaatggcttcttcgccattgatattattctcaccttcttcgtcgcttatctcgatagccactcctatcttctagttgacagtcctaagaaaatagcaataaggtacctttcgacgtggttcgctttcgatgtttgttccacagcaccatttcagccactaagcctcttgtttaactacaacggaagcgaactaggattcagaattcttagcatgctcaggttatggcgtctccggcgagttagctcgctatttgcaaggcttgagaaagatatccgtttcaactatttctggatacgttgcacaaaactcatttcggtcactttgttcgctatacattgtgctggatgtttcaactacctgattgcagatagatatcctaatccaagaaagacatggattggagctgtgtatccaaatttcaaagaagcaagtctatggaatagatatgtgactgctctttactggtccattacgacattaacgaccacgggatatggagattttcatgctgagaacccaagagaaatgctttttgacattttcttcatgatgttcaacctcggtttgacagcttacctcattggaaatatgaccaacctcgtcgttcattggactagccgaaccagaacctttagggattcagtgagagctgcttcagagtttgcttcaagaaatcaactcccacatgacatacaagatcaaatgttatcacacatttgcttaaagttcaaaacagagggcttgaaacaacaagagaccttgaacaatctgccaaaagcaatccggtcaagcattgcaaactatttattcttccccattgttcacaacatttacctctttcaaggagtttctcgtaacttcctctttcaattggtttcagatatagacgctgagtatttcccaccaaaagaagatataattctacaaaacgaagctcctactgatctttacattctggtgtcaggagcagtggacttcactgtctacgttgatggacatgatcagtttcaagggaaagcagtaattggagaaacatttggagaggttggagttttatactatagaccacaaccattcacagtaagaacaaccgagctatctcaaatactgcggataagcagaacatcgctgatgagtgcgatgcatgctcatgctgacgatggacgagtcatcatgaacaatctcttcatgaaacttagagggcaacagtcaatagcaatagatgattcgaatactagtggtcacgaaaacagagatttcaaaagcatgggatgggaagagtggagagattcaagaaaagatggctatggtttagatgttacgaatccgacttccgacactgctctaatggatgcgattcacaaggaagatactgaaatggttaagaagatacttaaggaacaaaagatagagagagccaaagtggaaagatcaagtagtgaaaccgctggaagaagttacgctaacgattcatcgaaaaaagatccatattgcagctcaagcaaccaaatcatcaagccatgcaaacgagaagaaaagagagttaccatccacatgatgtctgagagcaagaacgggaagttgatactcttaccatcatccatagaagagcttctaagacttgcaagtgagaagtttggaggctgcaacttcacaaagatcaccaatgcggacaacgctgagattgatgatttagatgtcatttgggatggtgatcatttgtatttttcatcaaattgagtttgaaaactcgacttcatttatagagcatgtatatctgcagataatgtatttttacccggtttcatagaaaagtctagattatc

SEQ ID NO: 8 - DNA, 1937 bp, Arabidopsis thaliana
  SUC2 (AT1G22710)
atcacaaaacaaaaacctcacttaaaaaaaaaaaacagaagacagaaaaaaacaaaaaaaaaaagaaaaagaaaaataaacaaattttcttttcttttttttcctctaaagtttctattttgtctattcgtgtttttttttttacttcctgataatgggagcctatgaaacagaaaaaccaactaaagatgcggcggctctagagacacagtcgccggaagatttcgatcaaccatctccgcttcgtaaaatcatctccgtcgcttccatcgccgccggtgtacagttcgggtgggccttacagctctctcttcttactccgtacgttcaactcctcggaatccctcacaaatggtcatctctcatttggctctgtggtccagtctccggcatgattgttcaaccaatcgtcggtttccacagtgaccgatgtaggtccaaatttggtcgccgtcgtcctttcatcgccaccggagccgcactcgtcgccgtcgctgttttcctcatcggttacgctgcggatttcggttataaaatgggagataaactcgaggagaaggttaaagttcgagctatagggatctttgctctcgggttctggatcctcgacgtagccaacaacaccttacaaggaccttgccgtgctttcttagccgacttagccgccggtgacgctaagagaacgcgagtcgcaaacgcgtttttctctttttttatggcggttggaaacgttttgggttatgcggctggttcgtacactaacctccacaagatgtttccgttcacaatgacaaaagcttgcgatatctattgcgccaatctcaagacttgtttcttcttatccatcactctcctcctcatcgtcaccgtcacgtctctttggtacgttaatgacaaacaatggtctccgccgccgcgaaatgccgacgacgacgagaagacctcaagcgttcctttgttcggtgaaatctttggagcattcaaagtcatgaaacgtcccatgtggatgcttctaatcgtcacggccttaaactggatcgcttggttcccatttcttttgttcgatactgattggatgggtcgtgaagtgttcggtggagattcagatggaaatgaacgatcgaagaaactatacagtcttggagtccaatctggtgcaatgggattgatgtttaactctatagttcttggtttcatgtcacttggtgttgaatggattggtcggaaattaggaggagctaaacggctttggggtattgtcaatttcatcctagccgctggtttggccatgacggttctcgttacgaaattcgccgaggatcaccggaaaaccgccggtgatttagccggaccgagcgctagtgttaaagctggagctttaagtctctttgctgttcttggtatcccattagctattactttcagtactccttttgctctagcatccatattctcaagctgctccggtgcaggccaaggactttcattaggagttttaaatttggcgattgtgataccacaaatgatagtctcactaggaggtggacctttcgacgccttgttcggcggtggaaacttaccggcgtttatcgttgcagcaattgcagcggcgattagtggagtattagcgttaaccgttttaccttcaccaccaccggacgcacccaaggcaacgaccatgggaggattccactagtttactattttttttttttaatgaaactcatcaaatacagtattactgtgcgaaactgcaaaaacccaaataaaagaaaagaatggataaaatgataaaatctaataaaccttgggtcgtctgtatttcacccttttttagttttttttcttctttttttataacttttgtcacttattttaatttgaaatagaatgttttttttttttgcccttaaataagatttgtgtgttgagtacaa

SEQ ID NO: 9 - DNA, 942 bp, Arabidopsis thaliana
  SUC2 (AT1G22710) promoter, vascular pattern
gcatgcaaaatagcacaccatttatgtttatattttcaaattatttaatacatttcaatatttcataagtgtgattttttttttttttgtcaatttcataagtgtgatttgtcatttgtattaaacaattgtatcgcgcagtacaaataaacagtgggagaggtgaaaatgcagttataaaactgtccaataatttactaacacatttaaatatctaaaaagagtgtttcaaaaaaaattcctttgaaataagaaaagtgatagatatttttacgctttcgtctgaaaataaaacaataatagtttattagaaaaatgttatcaccgaaaattattctagtgccactcgctcggatcgaaattcgaaagttatattctttctctttacctaatataaaaatcacaagaaaaatcaatccgaatatatctatcaacatagtatatgcccttacatattgtttctgacttttctctatccgaatttctcgcttcatggtttttttttaacatattctcatttaattttcattactgttatataactaaaagatggaaataaaataaagtgtctttgagaatcgaacgtccatatcagtaagatagtttgtgtgaaggtaaaatctaaaagatttaagttccaaaaacagaaaataatatattacgctaaaaaagaagaaaataattaaatacaaaacagaaaaaaataatatacgacagacacgtgtcacgaagataccctacgctatagacacagctctgttttctcttttctaagcctcaaggctctcttaacttcactgtctcctcttcggataatcctatccttctcttcctataaatacctctccactcttcctcttcctccaccactacaaccaccgcaacaaccaccaaaaaccctctcaaagaaatttcttttttttcttactttcttggtttgtc

SEQ ID NO: 10 - DNA, 1728 bp, Arabidopsis thaliana
  RBCS1A (ribulose-bisphosphate carboxylase) promoter (AT1G67090),green tissue pattern
ggtacccaaatttattatgtgttttttttccgtggtcgagattgtgtattattctttagttattacaagacttttagctaaaatttgaaagaatttactttaagaaaatcttaacatctgagataatttcagcaatagattatatttttcattactctagcagtatttttgcagatcaatcgcaacatatatggttgttagaaaaaatgcactatatatatatatattattttttcaattaaaagtgcatgatatataatatatatatatatatatatgtgtgtgtgtatatggtcaaagaaattcttatacaaatatacacgaacacatatatttgacaaaatcaaagtattacactaaacaatgagttggtgcatggccaaaacaaatatgtagattaaaaattccagcctccaaaaaaaaatccaagtgttgtaaagcattatatatatatagtagatcccaaatttttgtacaattccacactgatcgaatttttaaagttgaatatctgacgtaggatttttttaatgtcttacctgaccatttactaataacattcatacgttttcatttgaaatatcctctataattatattgaatttggcacataataagaaacctaattggtgatttattttactagtaaatttctggtgatgggctttctactagaaagctctcggaaaatcttggaccaaatccatattccatgacttcgattgttaaccctattagttttcacaaacatactatcaatatcattgcaacggaaaaggtacaagtaaaacattcaatccgatagggaagtgatgtaggaggttgggaagacaggcccagaaagagatttatctgacttgttttgtgtatagttttcaatgttcataaaggaagatggagacttgagaagttttttttggactttgtttagctttgttgggcgtttttttttttgatcaataactttgttgggcttatgatttgtaatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaattaacctcacacatatccacactcaaaatccaacggtgtagatcctagtccacttgaatctcatgtatcctagaccctccgatcactccaaagcttgttctcattgttgttatcattatatatagatgaccaaagcactagaccaaacctcagtcacacaaagagtaagcggccgc

SEQ ID NO: 11 - DNA, 1496 bp, Arabidopsis thaliana
  (AT1G62360) promoter, shoot meristem pattern
ggtaccaaacaggtacaggtgtcccatgcttattctcttgatagtaacatgtgtcctatgttgagtcaattctacgttcgaagaagtgctaacaattgttaatagcctcgtatattattctaattaaaatgcctcgatagatttggttagtggtctgaatgtgattggttattttttcaagtggcaagaggtctaccatctaatattacaatcaatcgaccaaaaaggtcgagaacatgataatggtggcaaatacaaatggttcattgttgtctaatataacaagccatcagttgtcactttttaaaaacaatacagaatacaagatacttttttttaaggtagaatgtgtgtttaatattttcgtttatataacaaataaacagttacatgttttactctatgattatatttatgacatttttcttcttcttaacaacatttttttcccataagaacatttacaatagtattaaaactttgattgcaatcaaatgttagatcacttattataaaattactaagactgctatcttttcctattgacaaaagcgaatccaatatatgttactgaaacaaatgcgtaaattatactatatggagatctatcggttaattattgagagaatctaagaaagtttttgagtacaacagtcctaataatatcttcacataccatataatatacatatatacatatacacaaatgtactctttaaaccaacatcagcatacgtatatcccatcaggaaacttagacttttgggaattcatggtatgaaaaccaaaaccaaatgacaacattcgatttgatactcccgacccatggtaaagaaataacaaattccaatatatctttcactggactttccgaggcacattccggttttctccatttcaagaaattgtcaaaaataaattgagatccggtttattacctcaaaaaagaagaagagaaattacaacattaatttccgaaaaggcataaatgagaaatcatatttcagcagaagaacacaaaagagttaagaacccacagatcacacaacctctgtccatgtctgctttttacacttttttaaaataagtttctcctaaaaagttatttcctatttataataatttccttagatttatcttcctggtctctcttctgctgcttccctctcccccataactatcactatttagaattttcaatgtggaaaaggaagctgattgttgaagcataaatcccgggagaccacttttgcattttcaaataattaaattaaaccatagatacacacacacagttacttactcttttagggtttcccaataaatttatagtactttaatgtgtttcatgatattgatgataaatgctagctgtatttacaatgggggctcctctttttttttctttatgttattttcattttttatttctcatagctatatagcaaagccaaagtgaataataatactagtgagagaaagagaagggcggccgc

SEQ ID NO: 12 - DNA, 2118 bp, Arabidopsis thaliana
  SCR1 (Scarecrow1, At3g54220) promoter, root specific pattern
ggataagggatagaggaagaggactttgtttatcagaaaccttttgatgggccttaatgggcctataaactgtaactctgtagcgctttgccaacaagagactttttaaggtttttgttgccaaacagatatttgcatttgggctatgtaatgttagaattattttataatgtatgctattgctagatattgtttaagtgcatttgtgatttacaaacatttcatttttattttggttttaatgagcatttctattatagagactttgatgttaataaatggtgttctaagatatattaaaatattttatatactttcttaaaattggataaattttgggaaaatccttaatatcagttaaattgaagataaagagtattaaaaaaaactatgtagtaaaatacatttcacattttttgtgtataatagtacatggtattcgttaagatcactcaaaaattaacaaattaagtctaaaagggcagaaaagactattcaaatatggacttggagaaagacattcagctttttacgctgagaaactttcatattgagccgtgtgtttgtgttgtgaagagaagtaataaaaaataatttgaagtgaaaaaggagaagaaaaaataagatcgtagaaagcgtggatggtttcttcttgggttcactgccatgcgattattaaattggccatggggctagtgtttgacgtacaaaagtctaaaaattgtcagtcaaacaggtccaaaactttgtaagaaaaataatataataatagcaaattttctaaaaattgttaaaaaaagaacaaaagggaaaagatgaggatgcagatgaaagcaaaatgtcaaacactagtttcagattttatcgggaactggggtttgacagttggtgtatgtatgtaatggcctctcatcaaaacatgtgcatctttttccttttttgttatttactgttttagctctacgtcttgtccaattcctctcaagtaaaatgcctttaatatgatactaatatacaaggggactaatgctttttcccttttcttatccttgttttgtctaaatctttacttggattcctttatttttctcctctctttagattagtacggtttaaggaataccatctttctaattttagcacaaaattgcaagttggtgccccatcttagtaagcacatcgtaccacactttgattgtgtgagagacttcttcatcccatctctcataccaaacctaaatcaaatgactagtggtgcaacctgctgactccatatgaccataactaataaatcggtttatgaatccaactcatgtagctctatagaatagaaacccattcatttcacataatgaactgaatctgacattttatttacatcatttactactcaattttgtaattagcaagatcatctttttcattattcaacaattttgatattccataatttattaactttgtcatacatcataatattctgaaattttgttatatattgtaccggttccacgaaatagagctctattattatagaccaaacaaacaaaatattatcttcttgtggttagttcgagagagaggtcaagaagaaacgaaatggatcggcaaacggaagacgtcaaacacacaacgacgaacattttccgatcacccacctaatctcttcccatttttattatttttcaaaactcaaattaattaagaagaaaaaaacagaaacagagagagaaagagttaagatgaatagagatagaaagagtcattaaatgtacgaagcgacattcacaataattcgaaaggtggaagacgacttagatacggccaggcttcactgtcctcctcgtcctcctcaattacccctaacccctttttccgggattcatctccaacccacatccttccaaattctcaccccctcactgagtttttgctttttctcctcatcggagatcgtgaagacgatcaagtaatttaagaatcccaccattgataaaagagtctagcttttctactaccaaacctttttctgtttggaaattttcgattttggatttaacccttttcttaccttatttataaccatgcaatctcacgaccaacaacccttcaatctcc

SEQ ID NO: 13 - DNA, 2498 bp, Arabidopsis thaliana
  SHR1 (Shortroot1, AT4G37650) promoter, root-specific pattern
ggacaaagaagcagagcgtggggtttcttctaataattgtagaagaaactgatcatgagaacatttgatctaccagagatggtgatgactcataagatgtaaatatctactgcattatgtctagcctaggctataatgtagatttgatcactttcttcattaattagtttggaattttagcatgatatagcatatatctaaatatgtccgaaactttcctacatactagaaaatatggagagttatgtaatgtaggtttgcttgttaatatacaaaataacatcatcatttagtttttagattttttattttattttttataatggtgctacgtacgtggcgatcaaattattccaattttgagacttcgggattttaaacgaaattaaacaatgggcatgagctcggggggatagacaagattaatgctttgtatcgagacaaacgagaaaatcatgatgagcctatgcattaagtgccgttggttaattagaggttcgcatatacataaaccagtagacatatggataaatatgaacacacacaccaaaaaagtgggaaatctaaataagtgtagagaataataagtcctcaggtgggagattcaaagagaggacaatgaagggtatatagactctaaacaaaaatggcatgacttagtggagagggttttaaattgaaacaagtaggattgaagaacaagaaaacaaagaagcatgccctagatttctgagataataattacacattgctgtttatataaggtaagagaatatgacacattggttggtttcttacgggtaaatgtgaagaaaaaaaaatagtaatatttgagaaaatctaaaatagtaaagaggtatatatggagaagaagagagaaaagggaaaaatagtggcagagaatggagagaggttaggaggcaaaggcaaatgtggagctttgatgatgttgatgcacgccgtcagcttttcttcacgcctgctcccactcactcacacctatgaacattctctctctattttataattatattcacatgtctctatgttactatgtaaatggtgaccacttaagtatttatatatcatgtatatatcttataggtatcatacaaaatggtcatgaaacttttgcaatttcaatctacttgttcattgtagatgctagcttttcacatgttttgaaaattagtctggatctgaaattctttaattagcattgttttgttggtcaacgtttaatttcttgattattgatgtcaaaaattcagagcgttcagaactcttacactaatttcttaaaaataatcgattaagagaaaatagagttttcatgcaccagtgttgatagtaacgtagtcgcggaatgtctaaaacgattatgagtttggtgttttgattggttagaattggtattagtaggacattctaacttttttgttagtctgttgatttaggatgcgtaaagagtctttttattttacaccagttgagacttgggatcgatagtacttgaaacacttggttggtttcatgtatttggcctatatataaacaaacatcgtaattatatacggatttttttcggaattttacgccatatctgtaagtatatataacatgcatgtcgttttcaaattcatatgatgaacgatccacgtaagtgctactactcctacaatattgcatgagagagatatgtatttataaattttattttgaagaagaaataagagggaaggttacttgggtggatcgatgtgaaaacaaaagaagaaaaagcgaaacccactaagccattacatgatatcgaccttcttatctttttcctctttattttatttttctcaggacttttttctacttaatgaaacctccaaactatctaactaatacactcccatgtagaataaagaaaattatataagatattgttgatattttgtaactagaaaatatatttgctctgtaatttttcgtaagttaaatcaacatttttcagtagaaacaaatattactgcaaaaagtaggatcattatttttgtccaaaatctcagttagctatagggttgtagtaaaaacaaaacacattcttgatttgccccaaaaaataaagagagagaagaatattgttcaaaagtggtctcttctctctctaattatgttttcactaaacccaattagattcaaacagtctacaaagtccaaaagataaacatgggacaacaattcgatgcaaaaaatcctcttttcatgctctttttttattctctagtcttttaaattactaataaaaactcacaaatccaccaaacccattctctacaactcaccttcatctagatttacccactcccaccgagaaacacaagaaaaaaaatatacatatataaatatacaagacaacacatgatgctgatgcaatatacacaacaaagtattaaatcttagatattgtgggtctccctttcttctattcattttcttattcattaaaa

SEQ ID NO: 14 - DNA, 1040 bp, Arabidopsis thaliana
  CYCD3 (AT5G67260) promoter, dividing tissue pattern
gtattaatttgatttgttctttctttcgttttcacttctttcatttgaataatatcctttgttgttttgttcaatctttcatttttgtattataacgaaactcaaaagaaggtattttgaccactaatgttgttgttaaagttttaaacatcctttttggataatttaatttttattgtaaaggcaagaaatacataaatatttaatctgctaatcgccttttttttcttgcctcgaatccgaattttaacatataaagaagcgagacaggttcgaattaaataatctctcttttaaagagatctttatagaatacataaaaaaaaatcaaactatttatagaaaaaataaaataaaaacgaatttttgctttcaatttatgtacattggaccacagaatctgcaaagctatgtatgtatcaagaaagacgtgactataataaataccacgaacaataaataattagattcttattacgaatcttttatgtattcccctaaaaaaaaccatgtcataattagtctcattaaaatacgattgtatgtaaaaaaaatcataccgtatgtagaaatatggacttcattctgtaaataaaagaaaaattaacgtgtcaagatctttgttaatcagtaattaaaatctaaaatgacttaactcaatccgcaacacataggactgggagcgtaatcatcacgatccgatcgggacatatgtttgtctccatcgtacggatagggtatatgagcgtgtttttcacgcgcatcattcgcgtgaactaagaatgctaatgaattcgttaattcgttaaaataaaataaaataaattatattagaaattaaaagaaaagaaagaaagaaaaaaaaaaaagacaaggcgtgagataataaaaccctttggctttctcatagagatttgtccggtctcttgttcctctttctcctttcttcactgtagaatccgtcgaccaaacaactagctccaatcgcataatcagcattgtagtttgcaatttcttcttccgtgaagaagaagaagatg

SEQ ID NO: 15 - DNA, 1730 bp, Arabidopsis thaliana
  AP1 (Apetela1, AT1G69120) promoter, floral meristem pattern
atatcatacatatatatagtatactatctttagactgatttctctatacactatcttttaacttatgtatcgtttcaaaactcaggacgtacatgtttaaatttggttatataaccacgaccatttcaagtatatatgtcataccataccagatttaatataacttctatgaagaaaatacataaagttggattaaaatgcaagtgacatctttttagcataggttcatttggcatagaagaaatatataactaaaaatgaactttaacttaaatagattttactatattacaattttttctttttacatggtctaatttatttttctaaaattagtataattgttgttttgatgaaacaataataccgtaagcaatagttgctaaaagatgtccaaatatttataaattacaaagtaaatcaaataaggaagaagacacgtggaaaacaccaaataagagaagaaatggaaaaaacagaaagaaattttttaacaagaaaaatcaattagtcctcaaacctgagatatttaaagtaatcaactaaaacaggaacacttgactaacaaagaaatttgaaacgtggtccaactttcacttaattatattgttttctctaaggcttatgcaatatatgccttaagcaaatgccgaatctgttttttttttttttgttattggatattgactgaaaataaggggttttttcacacttgaagatctcaaaagagaaaactattacaacggaaattcattgtaaaagaagtgattaagcaaattgagcaaaggtttttatgtggtttatttcattatatgattgacatcaaattgtatatatatggttgttttatttaacaatatatatggatataacgtacaaactaaatatgtttgattgacgaaaaaaaatatatgtatgtttgattaacaacatagcacatattcaactgatttttgtcctgatcatctacaacttaataagaacacacaacattgaacaaatctttgacaaaatactatttttgggtttgaaattttgaatacttacaattattcttctcgatcttcctctctttccttaaatcctgcgtacaaatccgtcgacgcaatacattacacagttgtcaattggttctcagctctaccaaaaacatctattgccaaaagaaaggtctatttgtacttcactgttacagctgagaacattaaatataataagcaaatttgataaaacaaagggttctcaccttattccaaaagaatagtgtaaaatagggtaatagagaaatgttaataaaaggaaattaaaaatagatattttggttggttcagattttgtttcgtagatctacagggaaatctccgccgtcaatgcaaagcgaaggtgacacttggggaaggaccagtggtccgtacaatgttacttacccatttctcttcacgagacgtcgataatcaaattgtttattttcatatttttaagtccgcagttttattaaaaaatcatggacccgacattagtacgagatataccaatgagaagtcgacacgcaaatcctaaagaaaccactgtggtttttgcaaacaagagaaaccagctttagcttttccctaaaaccactcttacccaaatctctccataaataaagatcccgagactcaaacacaagtctttttataaaggaaagaaagaaaaactttcctaattggttcataccaaagtctgagctc

SEQ ID NO: 16 - DNA, 1224 bp, Arabidopsis thaliana
  CUT1 (CUTICULAR 1, AT1G68530) promoter, epidermal pattern
gaattatattttactcttcgatatcggttgttgacgattaaccatgcaaaaaagaaacattaattgcgaatgtaaataacaaaacatgtaactcttgtagatatacatgtatcgacatttaaacccgaatatatatgtatacctataatttctctgattttcacgctacctgccacgtacatgggtgataggtccaaactcacaagtaaaagtttacgtacagtgaattcgtctttttgggtataaacgtacatttaatttacacgtaagaaaggattaccaattctttcatttatggtaccagacagagttaaggcaaacaagagaaacatatagagttttgatatgttttcttggataaatattaaattgatgcaatatttagggatggacacaaggtaatatatgccttttaaggtatatgtgctatatgaatcgtttcgcatgggtactaaaattatttgtccttactttatataaacaaattccaacaaaatcaagtttttgctaaaactagtttatttgcgggttatttaattacctatcatattacttgtaatatcattcgtatgttaacgggtaaaccaaaccaaaccggatattgaactattaaaaatcttgtaaatttgacacaaactaatgaatatctaaattatgttactgctatgataacgaccatttttgtttttgagaaccataatataaattacaggtacgtgacaagtactaagtatttatatccacctttagtcacagtaccaatattgcgcctaccgggcaacgtgaacgtgatcatcaaatcaaagtagttaccaaacgctttgatctcgataaaactaaaagctgacacgtcttgctgtttcttaatttatttctcttacaacgacaattttgagaaatatgaaatttttatatcgaaagggaacagtccttatcatttgctcccatcacttgcttttgtctagttacaactggaaatcgaagagaagtattacaaaaacatttttctcgtcatttataaaaaaatgacaaaaaattaaatagagagcaaagcaagagcgttgggtgacgttggtctcttcattaactcctctcatctaccccttcctctgttcgcctttatatccttcaccttccctctctcatcttcattaactcatcttcaaaaataccctaatcacattttgtaacaataatacaattatacattaaaactctccgacg

SEQ ID NO: 17 - DNA, 574 bp, Cauliflower mosaic virus
  CaMV 35S promoter, constitutive pattern
gcggattccattgcccagctatctgtcactttattgtgaagatagtgaaaaagaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctga

SEQ ID NO: 18 - DNA, 1923 bp, Arabidopsis thaliana
  G682 promoter
ttattaagtgctatgcgttaatcggcatctataaagtgttgcattgatgaacaaagtggatgcctaaactagacgtttaactaaatgtttagaatgaaatcttcatctcatctaaaaagtgttgcattgatgtaaaaagtggatgcccattagttcttggctttgaaatgtttttagaatgaaatcttcatcaatctccatatgtggttcaatccactcattttatcttttgttaaagatgttcttcaggccaatataatgatgaccatggatggtttgcaactcgcatataacacttctttatccgatggttacaagtattacatggctatagatagcttttgcatgcaacaaattatctatcaaagtttatgcatcctctaaaatatggtcattggcaagccactaaacgtatatattgtgacaatgtatgatgatatatttttatgtgttgactccgtttttcattaagtaatgaaacatgttgctctagattaccattttaatcgcaaacatatatgttgctctaccacgcattgcatcaaatgataagcttgaggacgcttcgacaaaaacatatttctccttcttcttactgaaccaaatgacaattgacaaaccccattaataaaatcggttagtgttaatgtgtcactcataatattaacttagtaaagaacaagaccacattaattaaatcaggtgttagtctgagaatatacgtttctcttcctcattccaaacttaaattcggaatttactgagaatatattgttagcactgaaaaaggttaagttgaaagtttgctagggatggcaattaaatatagtcttgccttggggatattcccttcgtggacttgtaagtttatttataggtcctcttatgtatatatagatgatctaacgatcgatatactatgaaaaaagttgttactagattttattgcaggtaatagtgttgaataacccgaaccaataaagcagttgtaacgaacacacgacacgttgcttactgcgaggaccactttgttttttgttttttttggctttaagccaatttaggaccaaatttgcatgattgaggatgcaagtatccaacccattttcatctttcgtagtgacactcatttacttttgtgatggacacgttatagtatatcttaaatattaaagagacatgattgggggatcattgttttaatttaaataatgtagattctattcttttcatggtattaatccaatttatagaaagttatgtgttattagcaattaagctaaatgatgaaaacaatcagtttagtgaaacaaactcgccgagaaaacatgaatggttgaaaatattattgtgttttacaaacgtacacgaggacaatagttttgtaagtttttcttaggcattgaaaaatgtttgatacaaaaagtaatgttaaaataattaaaaatgattttgtcttaatatatccaaaatttcaatctattatgaacaaagggagtataatttctgattgaatgaactggaatagcaatcagaaaagctttgaaaacaattgttgttgattattaatgatcttaattaacggcatgtatcaatatttatacaacttatgttccagtccaagccatcacaacggagtaaatgaagtcacgggtacttgtggtttttattggttgcaaacttgcaacttgcaaagatagctaacaataattaatataattaatgagaacaaaaccaatttagtaaattaaaatcctttaacatagaaaccgaccaaacccgttggaccgttggttacttgatttggttagttgctataaatagaaatgatggttcgtgtgcaaccttcaaaatacgaccactctctcagagtactctcttagtttctttcttcttcttctttgtaatacggtgccgtttgac

SEQ ID NO: 19 - DNA, 727 bp, Arabidopsis thaliana
  G1988 promoter, light responsive pattern
gtcacagtgtgattcgtaggaggggctctatggatatatcttaacagagcaattggatttagcttggctattcaaagacccttttatttaagaaaccatttttggaaagatttcaagatatagactattgttgtactagttgggatcagaacccaaacaggttcaccacagtttacaccttgtgttttgtatccttactccttagattataaattaagagtattatcttctgttttgtattcgacaaaagatcaatgtataaaagtttatataaaagactgcaacaatgcagaagaaatgtaatggaagcaaccaagaaaagaagaagaagcaatttgcaatgagaccaagtctctgaaaagacattagtgttgactaaatctccacgtcacaccaaaaggaagacgaatgacttggcggctagtgtaatagttttaaaaatgaccacataatctcaccagcctcaaaacctcacgacacgtcattctctccaattctacaaacaccattcatttcatttccctaaaaaattatggctcatgtaagtttctttgatgtgtgaactgtggaagagactactctcatcaaccatgaaccataaaaactccaccgctctttctctccctcaatcatttacatctcttccttaaatctctcttcccaccatcatcattccaaaccaattctctctcacttctttctggtgatcagagagatcgactcaatg

SEQ ID NO: 20 - DNA, 1000 bp, Arabidopsis thaliana
  WUS (WUSCHEL AT2G17950) promoter, meristem- specific pattern
catgctcctataaattcgagcatataattaactaaagaatactcaacatgttcataagtacacctgtcttcacactcgtttcacacattgtaattgacataaattttaatatcttttaaagcaaagcataagtaaaattatatagagtatttcttcaacacatttccggacgatatcaccacgaaaaatatcctatatcacctatgtgtcatgactcatgaccataaggaaggaaaaatgtatttttatgatcaaacggttttcactttttttattattcattctagaaaaaaattataatcgttttctttcattcctacgatacaataattcataacgatcttttagaatttttgttttgttttctgtgtgtatgggtaaatatctttcatacgtagacagatacataacaattaaagctggtgaagtaataattgtgaagagacagagaaattgaaaaaagagagtttgtttttcgtctaagtaaataccgatgatgccctcacctttctaattccctcctttgtccctttccattttcatgtttatttctaacttctctgtccctttcaatccttctccaccagatatcctctgattcgatcccccacaaattttttcataaaaattattattattctcactaaaactctcaagagtagtttcgatttttcctctgaataaaaaaaaatcaaataatatatttgacaagagaatttccttcctaaacataacattttatttgaatgttaattcaattgattatttttctttcttatatccttttctttggcaaaatttatttcttatatccaagcacatttttcaatagggttttgtatgtatgaaatcatatctacatgcataaaagaataatatagtgaataactgattttctagaatattgtaaaaccttctttttgggtccccatttatatatataactcaataccttactatctctctatatatatataatcttctctctcacacaaaacct

SEQ ID NO: 21 - DNA, 1000 bp, Arabidopsis thaliana
  CLV3 (CLAVATA3 AT2G27250) promoter, meristem- specific pattern
ctctcaataatattatccgattaaggtatcatccatattgttcaatataatttaagcatataactgtttccagattaaacaatataatttataagagcaactgtaatactttactttaaaagttttcaaatcagaatctcttttctttttctacaaatctggaaacaatttaatccatattgtttttgttttttttaccttctcatatttagatgctatttattattgtgacacaagcttagtttaatttcttgttggttacatttttttttctaaatacacaaatattatatgtttaatattatttcacacatttcttagaagaaatctaaatacatgaaattatatttattaaaaagaagtctaaactatatatgtatttaaatggaattttaaatatgggtagtaatatagaaacaccattgatatattagagtatgtgccggtgccgtatacatactgttgtattatagtaacctatatatggtatacctagtcaatgtgtacggtatttttcatatgtattagttgtgaacctccacagcatgttagacttaggaattaattataactgaaccagaactttgagtctaattatacccctgccgttttatacaactttgaatattgattccagtggctaatttaaataagttaaaggaaacaaaaactgcaagtaagtgaaaaatacacaattgtaagtaaatgttagatatttaatttatttatagctaaatcatgaacaagttcgtataagatctagatatatgtaccatataccttttctatgcccactatatatacttactacacataatatataaaccaaccaaaatatttgaatagttaataactatgatacacgtttaggacaaataaaattaaaaaataggagatcccatttctcgcccttgtaggcttacgctataaattggactgtccccttctcatttcattaccaaagtaaagaacagtttctatatttctctctttatctctc

SEQ ID NO: 22 - DNA, 1000 bp, Arabidopsis thaliana
  AP3 (APETALA 3, AT3G54340) promoter, floral meristem-specificpattern
accctgttttaaggcgttgaaatgaaaatacaagtctcttgtatctgaatttgtgttttaagcgaagaatgattgttcttgtgaagttgatacacaagttctttggatatcctatcagtataaaggataggtttccattttcgtgactcactcactgatttccattgcttgaaaattgatgatgaactaagatcaatccatgttagtttcaaaacaacagtaactgtggccaacttagttttgaaacaacactaactggtcgaagcaaaaagaaaaaagagtttcatcatatatctgatttgatggactgtttggagttaggaccaaacattatctacaaacaaagacttttctcctaacttgtgattccttcttaaaccctaggggtaatattctattttccaaggatctttagttaaaggcaaatccgggaaattattgtaatcatttggggaaacatataaaagatttgagttagatggaagtgacgattaatccaaacatatatatctctttcttcttatttcccaaattaacagacaaaagtagaatattggcttttaacaccaatataaaaacttgttcacacctaaacacttttgtttactttagggtaagtgtaaaaagccaaccaaatccacctgcactgatttgacgtttacaaacgccgttaagtttgtcaccgtctaaacaaaaacaaagtagaagctaacggagctccgttaataaattgacgaaaagcaaaccaagtttttagctttggtccccctcttttaccaagtgacaattgatttaagcagtgtcttgtaattatacaaccatcgatgtccgttgatttaaacagtgtcttgtaattaaaaaaatcagtttacataaatggaaaatttatcacttagttttcatcaacttctgaacttacctttcatggattaggcaatactttccatttttagtaactcaagtggaccctttacttcttcaactccatctctctctttct

SEQ ID NO: 23 - DNA, 1000 bp, Arabidopsis thaliana
  PI (PISTILLATA, AT5G20240) promoter, floral meristem-specificpattern
caaagacattttccccataacaatgtaagcatgataaagtctaatggttataccaaggtctttggtaattacactgctcctttttcttttttttttttcctttttctaaaccatctgaatttaactttattttatttacttcatttgtcaatgtgatatcttcaactttcaataatttaatatgtttgattttttagtgtagctacgatattcgataagacctatatatgacatagagttcttgaaatagcaagtcttggttttgccaaagtaagaacctgtaaaaataaaagcaattctttacagagatttttggttttaaatctacaaagttgcaaaactcgttgctttcatttgatttaatggttagttttcgagatagacaaaatgggaaatttatttgcaaaatgatttagttgcaaaatcattgggacactatcttatttcacgttttatataattgatgacataataagatagtttcccaaagtaattaattgatgatatatgtggttggataagaaattatggtattatcatgtttgcctctcaaatttaaatttaattaattatatacatacacgagtaagctaaataaaagtttgaccacatttcatatgaagaattttatctttccagatatctagaatttgttttctctacacagttcattgaagaaaacatagtacggaagagaccagaggttaattaaacgacactttaacctatcacgagagagactgagatgatcaaatcaaatgaaagaaaataaacatcaatcacatgcaaagagtgttcattaagcaaaatcactaagtttgtttttactttattttattacgttacttcaagttttttttttatcttcttggtactgtaaaaaaaggagagaaaatagagttggctatgtgtaataagcgaaccaaaagcaagccttccatgactgtgccctcaagaaagtagctttgttttcaatcccaaactgtcaaagtctctcttc

SEQ ID NO: 24 - DNA, 1000 bp, Arabidopsis thaliana
  SPA1 (SUPPRESSOR OF PHYA-105, AT2G46340) promoter, light-induced
ttttttttttcttgtggtcattcatagtaactaattaaatcaatttgcaattttttgagtggtattggtatattttccatcttaaagactcgatggtagagaaataagattcctaaattcattagtcacaagatcgaccaatttttatttttatttttcttatctcaaatgtttcgttttgagaacaaccaaacatttaaccattgataaaattggccaatggctaaaatgaatgccttttcattgtttttttgacatcctaaaataaatgcttaactaatgggtttatggaaaacaaaagttgtgtccaagttataagcgaagcagcccataaaagtattaaaatgggctgagcccaataactgataggtggagtatattagtttaaattgtgtttgttgtacaaagtttcccaaatgggtttccaaaatgggctgagcccaaaaagtgttgttataggtgagggtatattagtttattcgcaaaaaaagcgtggcttaataatctccacatctttcacgtgacccccctcgaatggcgaatagattaagcgacgtcgacgtgttttaatctcctcctcttaacaaatatccctaaagattacttcccccatgcaacactcttaatacccacgcttttcctctttatttaccatcctacccctatactcacacatatcgtttaaccagagaggaaaaacctgagtggcataatccgtaaataagaccaaaaaacaggaccatctctaaagctgatgtcactaatcaaaagattatgaacgtaatctcgtgtcataatccttttgacctcttacttaaacaaaacaagtataataattgggcagccaaaataaaaaatctttctctctttttgctataaagtaatgaaaaaattaaataaaataatgttcaaaaaattgtccgccagaataaagaaaatttgatatgtggcttcaaagcctaaattaatcattaaaataaaaaatcagaaaataaggaat

SEQ ID NO: 25 - DNA, 1000 bp, Arabidopsis thaliana
  ELIP (early light-inducible protein AT3G22840) promoter, light induced
aagttattgacattttgaaacgaccgtaaatattaccaaaaaactgacggagttaggatcggccacgtagaaagggacaaagagagaacagtcacggactcggccagactaagtatgggcctgtctgaatccaaactcagctaagttccaaaagcataaagagagatgtgtaatgaaatgaacgtattctagaaacgaaagcaatgttatgctttgtttttgagccacatgtttttgggagatggagagaatcttttttacgtttttaacctaacccacttggcacttggccaaaaaagtgagaagaaactgtggcgaatgagtaggccacgccatggactttgttccttgtccttcaaaagttaaatttatgttatgcgtggggacaatctaagcaacgtggttcctttaaatatcgcagcttcctcttttacacttttggagcctacgtgttttgttttggaccggccaaatacacgagtcagtcagtttagaaataatttggatgtccaaaaatcttggagatccaaataaaataattagcatgttttagttcataagaatatgaaatgtagataaactgtccatattaatttttccatagaattggctttttatcgaggtgatgtacttaatgactttgttgattactactcgtataacaataaagaatatgatactatgtgagacttataatgaatttggtgtgtgttaattaatccagttgaaacagtttaataacaaatcagaataaaaattgtagtaagaaaatttgaacgctgatccttcaacctagatagtgaacctttcaaatactatatgattcacgtgtaatgtttttgaccgttggttatttttgtgtgaactatattaacttatcaatatcgaaaggctaaataagtaaataactaaaagaaagttcaggaaacaactcgacctaatgacctatcatttctgatcacccgtcctataaatacatacgtaagatcattcgttactc

SEQ ID NO: 26 - DNA, 1000 bp, Arabidopsis thaliana
  HY5 (elongated hypocotyl 5 AT5G11260) promoter, light-induced
gatgaaaaaccgccagtagcagcacctgagaactcctcacctgatccaagtcctgtggaatcattatcagctgaagatgttgtggtcgaacctaaggaaaaagtaaaagatgaagcagttcatgtgatgcaacagagatggtctgctcagaaaagagtgaagaaagctcacattgaaacgctagagaaagtttacagaagatcaaaacgacccactgtaaggattctccttttacatttgaatcaatttctatgttacttgaatgctctatctcacatatgatcatgtttgatgatgctgtgaatagaatgctgtggttagcagcattgttcaagtgaccaatcttccaaggaagcgagttttgaagtggttcgaagataaaagagcagaagacggagttccagataagcgagctccatatcaagctccggtttgatctaatgttaacgttgagatggcaatgatttgtatacttgattctcagaaactcatcaacattgtcgtcaaggacaagtttttttggtgatacgaggagtgtttatagtagtagattctgtccaatggtgtggctggatatgttggactatgaaattttaggatatcttgtattcagtttttagttatttccttgctgagattgtgtcttgtagaaaaccgtttcaactttgtttggtttatggcggctataaagtttaattttaatgcatgacaaaaacaaatcaccaaaaataaaataaattactttcacgacacttttgaaagcactgccctaggcgtgggccatgtgacagaatgaaagaactcagaccaaacttttctgtccaaggacaggaatggggcccacccaattagctcccctatccattattcaccgtaagatgctaaccagatctaacggctaaaatccacccacgttccaatctcaattgcctttggatccttgtatttcctcaaggctcacctttctccacgattcactctcgatatccgttcgattctt

SEQ ID NO: 27 - DNA, 707 bp, Arabidopsis thaliana
  prAT1G16420 disease-inducible promoter
atattgactattggaccttacatattccgattgtgtttgctatttactacggactaccgttttgtttttgtctcactttgataattggtgaatttttcatttttggatcagctaagtgcgacccaaacagaaattcaaaagtcaagagcataattttagtttcctaaaataggaattaatttatggatcttagattcacaaccgcatgtggaataattagtaaagaaaatgccagcctttttattattttttctttgtcaacaagctaatgccaactcttttaaaatgaatgaaacctactcatataattcctttttggccacccgtagactattccagacgattaacttaatgacactcatgtttttttccttaataatagaccatagtccattaacactttctttttattaagagtaacatgagagttatattaacatttgataataaaaacgacaatggaaatagccgccactcaaaaaagaaagaccaaggaaaaaattaaaaatgagacgtaaaaggccaataacagcaaaccacacaaagtttctcttaggagaagaggcaaaataacagtcaagcatgttggtccgtcttcagacctttcgtggtagtttaaatgctaagtctttcgtttataaataaagtgaaaaataaatgcaattcataaagaaaaaacaaaggtataattcattcattcgtcaatg

SEQ ID NO: 28 - DNA, 1203 bp, Arabidopsis thaliana
  prAT1G26380 disease-inducible promoter
gtttagtggtaaaggctattcggagtaaccttcagcactcaggttcaaatgtcaagaaaagctaatttttcataatatatatatatatatatatatatatatatatatatatatatatatatattttttactaaaacaaatctgaaatttaaattattatcataaagctacttctttattttagttacttgtagtcttaaaatacttagggacggtttatattaagtcacataaaaatcatgaatcattctattatatactaaaagtataaaccagaatttatcagtagtatcatagttcatcaagaatatcacatttcatatcaaactttcagtatatatacgaatgtctgataaataagttagaaaaaaaaactaaaataattgtgaagcataacaattcacaaatcaaaattaacttgaaaaacatctaattaaaacaaaacaaaaaaaaaagatagttacatgcgtaaataggtttaagtctacataaattaatataacagtagacgcagacacaatttaatggtggtctgatttaacgatgacggataggatcgacatttctactataagaaaagtcaatcgcacttttaaattaaaagataagttatgtatcaaaatttctcggccatcttaaaataatgggaaaataataatatagtcattagtattttacaacaacgtagccttataaaatttgaattcaacgaggggggacaaagaaaacaaaggattcaaagagaagagagaggaaaattcagtgcattctacaaatacatttggcataaaattcaacaatacttaatcgcaattatttcaattagtagatagctaggtttggtcaaaatatgaatgaagtcttaccttaggtttccatttataaaatctcgtggtcacttaaaaaatctctgtattcaactacctaaaatgatcatttgaaataaagaagttcagttgatgcgactcaccccctgatctaaattatgaaagtcatttcccctgtactatacgtattacgtacgttgtaatttcataactttgttcaaaataaacagctacttgacgaaaagtcaaaccaaattcaaaagtacaccgatatggaaaaaatggtcaagattgtcaagttgaaattattgtctccatatatattggtattctataaattacaaagtagaggcataatgaaccaaacagcaaaatg

SEQ ID NO: 29 - DNA, 709 bp, Arabidopsis thaliana
  prAT1G26420 disease-inducible promoter
atattcgtttttataagaaatgaaatgaatacagataatatattagtaggtgctacaatctccaaagtttgcaaaaatttgagtttcttctattcggtaagaaactctaatatcaaaaaatctaaatcgctagaaggagggatcgaacctccgaccttgtggttaacagccacacgctctaaccaactgagctattccagcttttgttaatatgtgttagttaacttttatatatcttaccataacaaaaaaaaaattcaacaaagattaatcgtgcaccgggggtcggggggatatttttaagataacattcaacaaagattaatcgtattcaactttattaaattctctatctgtattcaacttttttccactattgaaatttgcaatatatatactagatagaaactacaataatataatcccaaaacatgcatgtcaaatagcgaagtaattccatagtcaatccctgatcccagtcatgactcatgacaacgacttctctgtaatagcgttggaaatcatttccactagcttgttcaaaccaaacatctacatgttgactaaagaaatttcaaaataaacctgcaaaaaccaaattcaaagtaaacctatatgacaaaacagaagtcaattatatattaatttctctctattctataaactaaaatcctaatagagacgtaagacaaaattaaataaaaatatg

SEQ ID NO: 30 - DNA, 1203 bp, Arabidopsis thaliana
  prAT1G28190 disease-inducible promoter
aggcgggagtggcaattcaaaaccaaaataaagtaatgaagaagcgagtcaaagtaaggcaacataattacgtacactatttgatgagtcaaacctacatctttctatgaccaaatttgagaggtggctacttctcttaggctttgcaacttgaaaaatatgttccgcatctaatctaataataggatgttgtcatgttcatatgtttcgttgaaaaatattaattctaattaaaactcgtgttaacatcaggattggatttttatgttcatagtgataattaaatatctccaagattagtgtaacaacaacaaaaaaaagaataatacttatatcataatctcaggatgatcatgattgctcaactaagtagggattgggtcaatcactggttaaaagagaagaaaaaggtgaaatgattattctagagttttcattaaagttgaaataccttaaaaccaagattgaaatgtctaaaaagaacactcgatacttccttttaattccaccggtctaagtcttctttcattttatatatttgtataacctaatgccgtcacgcacgttaaacatagtcaaattctttattcatatattattattttaatcgcttcttaatcacagttaacatacttggaccaaacctagtccatatatccaacttttaaagcatggatgtccgataacatagttgaaacatatacatataaggtgtgcataaaaatataaataatatgtatgcatgaaaaaaaagaaaaatacaaaacattactaaacgaatggaataaaaaaaatctgtatattaatagatgagtatacttattagtaaaaatttattttaaagtaaattgaaattagccaaaaagataaagtgaaaaagatgtaaaaaattgaatttttcttgaaagccaaaaattatttgtttgggcccctatttgtttaccaaataaaatgaaatgaaggagaactcatatatttgaatatgaaaattgaaaacaaacacatttttaaagggagataaattcttttgtatgtaaaatactctcagtgtatatatatacaaaccaaaacttcatttcatttgttcacactgaaactccaaaactctatctctctctctcctccacacaccaatttcttcatgaccttcttcttctagcagagaagattaaagaaacccccaaatccagcttatactaaatg

SEQ ID NO: 31 - DNA, 700 bp, Arabidopsis thaliana
  prAT1G56060 disease-inducible promoter
ttgtaattgtcagtcatgcattaggttcacttacatggtaacggattatttgtggtgttgttgatttagtaaattggaatgttgaagaatgcagccaggtcccttgaatagtgggagctccttaaaaaatattccaagtcgatagtgtttagaagatgcgttcggtggctatttcctaaggaaacccacattgctcatttatcacttagtttaattatctcatctataaataaaacgtccagtttggacaccaaatcacgaatcacttattaatctttggtgatcttgtgtattagttttaagaggatgtcatttagagagttaataagctgattcgtgatttaacaccattggcccaaatacatgattgattatgggtcgtacaaggcaacaataaggttggttacttatcaaaacaccggtgagaacacgtccattgactcgttatgaagtgttttgacctggaaccttcagagacgaccagaatcaaggacgcctcctacttttatttgaaacgcgttgtgtcgtgttcggtctggctagaaaccggaagtttctacgaacacacctcctaacaaattcaatatcttaaaccggaagataacgtcagaaaatgtataaatatacactttgaattgaagcaattcacaaaaatcattcatcatctctcttaactcatcattacacagacatagcaatg

SEQ ID NO: 32 - DNA, 1203 bp, Arabidopsis thaliana
  prAT1G61560 disease-inducible promoter
gttcttaagaacacatttgaaccaatcatcgaaatatatggtggaaatatatttctaccaaagatttgttttttaaccaaataattgaacacaacgctaactgaatattttataaaactgatacagatttattaaaaattttgctaataaacattataaaattgcttttaccaattataggatgtattgtatctcttggactttaatatcgttgacgatcttgacaaataaaaagctggcgtttcattaaattggtatttacaatgagaaaaatgtgtgggatccaaaactggatataggattcgctttactgtatctggatccgaaactctaaaatatgatgtttcttatgatctggattttttcaaactataaatgatttctgaatttccgtgtatgtcaaccaatatttaaaaacagattatatttcttaaatatttttcagattttttgaaaaatattctgtaaattacaaatgcaaataaattatttaagaccgttaaggatcaaatatgtttttagtttattcaaatctctcgttctctcatatcgctattttgtacttagtttagataaaagtattaagtttggcttcttagaatttgatgtttctttttttcgtttttgctatcgacttttgttttttttttttttactattttggtgttatttatctattccttaaattttggttttgtttctgatacttatattttctgtaatttatgtcgcaacttcaaaaataatataaatactttacattgatattaaaaaaaaaaaattgagctctaatgacctttggagctcatgctcatctatggaaaaaaaaatccatgcaggaagaaccaagaggaaacggtatacaaaataatattaaataaataattatttgtcaataaaataaataaaaactcaccaaagtacataacagttcacacagcatgtttttagaaaagatcatatactattggtttcaaagtctttgactttgaatgtttgaacttttcaaggttcgcctactcgccggtcgtctcctttaaccttcgtctctgtgtttatataagaacatatacgtatttgatgattacaaaaagacaattctttatccttctttagatttctgtggaaagttcactatttattaggagagacaatttcaaaaaggaaagctttttgcttggaactgttctgtgagttctaatg

SEQ ID NO: 33 - DNA, 1219 bp, Arabidopsis thaliana
  prAT5G24090 disease-inducible promoter
aaatggtccagttttggcccaaatatttaacaacatttgggttacgagtatttgccctttacaaatggatcaacaatctccctggatcaatatttagtggccggtttcatgaatcaacatattctttttttttttttgtctaaagaatcaacatattctaaatcaccaaaacactttggtcaacaattttcgacaatatatggaaattaggttggattatcatgcgacttttttctgattaattttatgtatttttaatttacgatgtaattcggactactaatttgtattatgataactttacattttccatactactcaagtccaagtaaaatactattgtatatatatctttggattttacataaattaatggggaggcctaataaaatatactcggagtatatcatttgactttgaaatttatcgagtcaaatcaatgattgtatttttggtaaaaacaattattatgaagactttgaaagtttttaatgattttaatttcaaaaattagtaaatgctggtctggttatccatccattggaagagaaaataagaccttttcaaagctagttgataaaaaaagttctcggtcctatccctcatcttataaagaaattattaatacgtttagggattcaattcacagaagatttaaaaacaaatggaaaataggatattaccataataattatggttcaacaacaatttcgatttctaatttgaataatggaaatttagatcaaaaatagttccgactcatagataaattgaaatgtgccaaatgtcacgtaaaccagcaagaggacaaagtcaacaccacaagagacgacgacgagcacagtgtgaggttatgatatataccctctgcgagactgcgactgctattactgatttgatcccaagttttttttttttttttgaaatttattttttctttatacacaattacatagtggtaagagattctagatggcttcttaatgtttgagatttatatctagtttaagtaggaaagctatattatttgaagaaagaaaaaaacaaccaatcaaagtcatgcaatgtgtgtgagagacattataacatacatagataagatataaaaattaaagcaaacaaaagtcatattttacttcttttataaaaaaagaagttaagcaataacaaacaaacacataaccacaaagaagacaaaacatctttaaccaaaaacatg

SEQ ID NO: 34 - DNA, 1206 bp, Arabidopsis thaliana
  prAT5G62150 disease-inducible promoter
ggtatatgcacgacaggacaaccgatacaatgacagttggttccaaaaaaaaagtttaatcctaaatatatgaataatcgaatcgatcaataacacgttgacaaaaaacgaacaaataatcacactgatgaaccactttataatgaacagagaattttgtaatctgaaaattttgaaagtcaagaggttaatcaagtaattatagaaaggtagttgtaacgttggcttttgtggaactaataacttacgtgtctttaaacggcggctactttggaaggctacgtttcttaatttgaacctcattttctccattttccttcgtttatacgatatctttttcaaaaaagtgacccaataaccacacatataacatatttagtataactttgaatataaacgaatcaatgatatctgaattttattttgattttgatcttgatttttgttgttttttgtcgaggctattgccttgccactttggatgaaggaacccggctaaggtaagaccccctgcctaatattagcctccggcgaattttgcactcagaaattacattatgttatagttttggaattttagtttaaatttgtaaaagtattaaaacaattggtcaactattatattaattagctcaagagtgctttcaaaaacatatcttaaatttaataaagaaatattccaatatcttaaccagtactaaaagagaagatcagaaaatttcttataaaactttaatctaataaaatcatctacgactctaccattcaatatttttttgttattgttttatttacatatttcttttaatatttacatatctcttttcctttttgctaaaaaaaagttggcataaaaattactaaattttaagcgtaaaaaaataaaattaattattgtctattgccatttttggaggatggatatgatttggaggaatagttaaagaaagtgctaaaatctcctttagtgagtcacaaccgttgaccttcaccgcaaggcacaagagaccaagtctctaacccaacacaacacaaaacccataaactgaaaagactaacctaccctatcttgccatataaatccctctcgagcaacgcatgttaaataaacctaatttatacattcattctcaaagtcaaaaggagacagggagagagagagagagagagagaattcaaagcgttttttttttataaattaaaggc

SEQ ID NO: 35 - DNA, 1203 bp, Arabidopsis thaliana
  prAT2G32210 disease-inducible promoter
tcttttctttttttccaatcctttggaaaattttaggacaatttgttattgtggctcaacgtaaccgggaccgcacgtttgataagttattgtttttttttttgttaaatgcagtcttgcgaccatgttggcctgttgcgtcctcgacgcatgcattttctgagttcggaggaagcagtcgattgtttattaaatttgacacttcatgcaaatatttttcattgtattttgcaagtagtttttgggattcgattagcttataacatttggacctcctatttgttgatttaattatttattactactttttttattttattacaacttagtttgtaatagtaaaatttattctactttgtgcccaaaactgtcaattgttgttcatttaaatttctggaattagatttagatacgctaaattaaatcacattttgcgtttatattcccacctaatttgaaaccagcaattattcttcttcataagaaaactttcatgtgctctctcgaaaataaagttttactctaataacaacacgcactttggacgagataaagcgattcaagtaaaattattatggttcaaactattatctaagatccgtttgtgtaaaacataacttctaataacatatatattctgatctacttttgttagtttttattagttaatataagcgttaattattgttctctcttttgcttaacatgtaaatattcccatggagaaagaagtacactttttgctttgagaaagaataaaaaaactctttattattactaaatcaagaaaaatataaaaccatgctgcatttatcatcattttacatattctatatataaggttctctaattcgtatacttttgtaaatagagaacaacgaattaaaagaccaaatcgatagcatcttacctttttgttgatatttattaagagaaaaaatcaacacattgttattcttcaagagaataaatttctggagacttagcagttaatgcaatctgacctcacgcgttttttttttttgacctcacgcggttttactaaaccgaccttcgtttacttcccttacctctctatatatatatctctatcttcatttgcatatttcaattcatttcataatcatacacctctctacatttgttactactttcttctaacttgttttcaaagagaaatcacaatctatctgttccaagatg

SEQ ID NO: 36 - DNA, 1203 bp, Arabidopsis thaliana
  prAT2G35930 disease-inducible promoter
tgattttgaaaaataaagagaggataataccattttggcaaaattgtatattgtgatattcatgaagaccataaacaaaattatcctcgaacgagatgccatatcatcaaagtcgagaaacgatgggttagagatttgtagttttgttgatcgtgtgatgttgccaaactacgggttcgaaagtcttaaagacctttataaaggaaatgcgtcgagtattttgctcgagagcctcggttgggtcagtgtcaacatgttgagtgataagctcgaggacattagtatctatgaaagtggaaatggttatgaagtacatgtatgagggtattggttacagggatatgaaagacccatgtgcgttatatggttatagtattcatatatcaatagtaaagcgacatcacgtgatattttttcttaatgtatcaaaattccaccaatttctattttcatttttgattaataaatacatttcactttgatagtatttagtaaatattgatgaataatcaacagaaacaaggccgtacgagaaaagttgtatactctcactattatattttattttacgacacaacaaaatggaaaatcttaagtcaaaacgggtggcaaaaatgtgtaaaaagagaggaagaatcaatcaattaaaagacacaaagcagacagtagacactcttgtcttcaccaccgccacgatcgcgaccaaatggctctctttatattttatcacaattttcttatccgtttgttacaatctctctttgaaaagtcaaaccttttcatacgtctcacgtgttcttttttcttcacccaactcatcagcgaaaataaaaggtcaaatctatgttcctcgttcgttccttctatgagtaaataatactaataaactttattaaataggggcagattttttctttttttagcatatagttatagggacagctttacaaagagtgtgtaataactaataatatttgtattttccgtgttttgacttttttaataattgtgaatttttgacatctcctttatatttaaacccaacctcctttctctcttcctcctaacttattcaaaccaattcacatcttcccaaacccaactactacaacttgtattaagaaaaagatatattcccttagcttctttgatcaatatattcgtcagggttctcgtcaaagtcctcagcatcttcatcatatg

SEQ ID NO: 37 - DNA, 1203 bp, Arabidopsis thaliana
  prAT2G35980 disease-inducible promoter
ggacgagcagaatagtggggttttcacgttgactatatgcgttgatggacgcgtgaggtggaaggtcgggactcttactatagggaattatcatctccatgttcgttgtcaggcgttcataaaccaggctgataaagcagccggagttcatgtcggcgaaaacaccgttaagtacacgttgatcaataagtgcagtgtcaatttttaggaggtaaaaggacacaactttgacgccgtcaatgtttgcttttactctttcttcttcttttcatttttcttctactcggataatgtttttgcttctatatcgtttaattttctacctttttctgctgatcatataacatacataaaatcaaaaaatttaatatgataaattcatgttaaaagaacttgccaaataaaaaccgatacagaattttcttgtaaaacattcttcaatcttttttatttatttttttaactttaaatttccacttaaattaaaataaagaggattacaaggttaaaaaccccaacatggccgcaggcctaaaaaagaaagataaattcttcaaatttatatttacgtaaaccttcaaatttaataaattttaaaaaaccagacataattatgttgagagcatcagcaacggtaatttctcaacttccgtttctcaatatatgtgtaaacataaataaaagtgtgaacacaaatattattaatatttaattgaaaagtattttaattaaaccaatgattgaatgacaactgtgagaaacgttgtcgataagtttctcttggtttctctaaagagaaactttcccttacctctctcctcctttggacatttcttttcttattttattggtgagagattttatgagaaactcccgttggagttggtctgacacaggcggccctaggttctagaacggtataacgagaaaccaacgtacgacgagagaccaaaaagtaatattaacatatacgatcttacaaaaagtactcattattggaagtttggggcaacatcacaagcctacaattgcataatattctttggtcaattattcaatcaaatcaccgcgtaacgtgaccttaccttaatctaataagttgaccaacgcataaatgaaagtgtatataaagatgacttacataaacctcttagccatatatccattcattccaatataattctccacaaaattactatcatg

SEQ ID NO: 38 - DNA, 1203 bp, Arabidopsis thaliana
  prAT3G18250 disease-inducible promoter
attaaggaactttaatttttccaaaaatctgaaacatactatacccaaaaatcgatgctcacacctagctagttcaaactgcgtacgtcaaaatatgtggtatggacgtaaagtattttataagttttgttagaaagttagatacttataaaataaaaaaattgatatttttttggtaaaaaatggaagtttggtattttttggcagttgtatacataaataaaaatatatattacggagttatatattcttgtttggtcaaatgtttcggaagcttttagattacgaaattacataatccatgatgaattaaatttggttagtggtagaagaataagacgccaaaagaaaaaagggaagaacattgggttacgtcgaaagtcctttgataagaatttgattttgcatagtcaaatttggaccaacaacaaaaatggagtccacgtgaaatagaagagagagacttaatagcttctcatgcataaagttatgaacaatcaatgataaccaatgataaataactaaacagtacaaaatctctgtatttttattggatttaacaaaggccatgaacaatcacttcaatatttataatttttttaaaaagacgaggcaattcaagctgatttatacattatgattaatgataattatattatggcatttgacttttctcacgctaatgtaaatggtcaaaaatccttatcttcaactaaacttcctcgacaaaaccttaactagaactatgtatgtaatttatcaaatattgaactttaaatttctttaaaagcataatataatcttgacctataaaattagcctagcctttcgaattacaagttttatatttttaaagaaacataatcttcaactagaaattattataaaccgggtcctatcttcatctaatatacgtgattccatcaaaattccgcagtcaaatgtgtttagttgagaggaggagaatagtagactaaaatggatgactttctggtaagtcttgtattgcagtcttttctatattttaacaaataagtcttcttttcttaaaaagaaaaatttaattacaaagaaatcttactactgaacgaataatttatcaaaagtcagtggccttacgtacgaatttatcggctataagtaggaaagcttttcacgttaaacaacttaatctcatccacattaactagagaaagagagatg

SEQ ID NO: 39 - DNA, 1203 bp, Arabidopsis thaliana
  prAT3G63380 disease-inducible promoter
cttaatttgttgcatgctgagagctttgttattactagttaccgttatgagtgagcttaattagttgtaatcttcccttctacttattcctgaaccattgtttagtttcataagatgatggttagtctatttattctcgagcaagccattgtatttgttggttgggattttgttaagcttgctactgcttttggtgcaaacactcttgtttctttcttatccacatacagtaactcgtgatctaatgtatgtcactgagcatctctgtgtatgtatgtatatgatttgaggagtttctcgacattcacttcatcgcttgtatcggaattttatgccaaatctggtctcacctcacgtctagttttagtggaggtcaacttattttgtttgtttataaaaataaagtcaactgcttttatgtcttgctgtcgtcacatgaacaaagaaagagatgacagcgaaaatggaaagtgctatgaagtttcggtgatcaaactcataatgccttcgtcgtacacgctgctccattttattatttctaaacatagatttgtggaaaagtagacggattttgagtttgagtagtataaatttcacttgatagatattataccaaaccaagttggtgtggaagattcaatctaaatcctttttttcttctttcgctttactatattcgccatttcttttcatttgtccgtgcggactaagcaagttggtacacgcacgctattcctcctcattctacaatgacggcttgtccacaccattacatcatacggctgctttagaaattactaccaaaatctcagattaactctctcttttagggcacagtggttccaaaccgaaacacgagacaacagataagtcaaaacacaagtacaaaaggatgtgcggcccatgtcctttgatcgacatgacttcgtgtttacaacgtttctgagtggttccaccgcctgaaaatattaaactaggacaagttactttacacgagatatttaatattttaaagcagataaatagcaatcaaaaggcggccaatacggatttaaatagtaccacgaagcttagggtattagagagagagaaatcattctttgcgagtcttaagtgtcttacaagtaacaccacatttagttgagagagagcgagactcttttccttctaaaaactctctttctttcacacaaataaagcatg

SEQ ID NO: 40 - DNA, 910 bp, Arabidopsis thaliana
  prAT4G01010 disease-inducible promoter
agcgtcatttatcgttcttttatctacaaaaaaaaacaagatttcatgagtcagacaagacaaaagccataaacaacacaaaagtcctgcaagattgctcacttgggcggttgggcctatatgaaaatttactatctcaaaatttaactaagagcccataaaaactgtaagatctttgctttaaatcacattgtcttcatagattttgtgattcatggtagatttgtgaagatctttggtcatctttttgtttacatttggcttattgagtgtagagtgtgaacaatctgctatgaagttggtaaaattctttggatccaaacctttaaatcgtagcatatttactatttcacactgctaggatctgtttatagctgatgcattactggaatctatgatgtttactgtgttggtctgatatataacaagttctgaattttaaaatcaaattcaatatcgaattgggcttttaattaaggttaggcccatcaaatgtttttgactctttaataattccctctcttttgtcttttattgtaaaatgcacacaatttcgaagaaactgccaatggagtctccattttccagcgccatgccaagcttgactctacgatgatagcttatttgactgcttcaaaaaaaaacttcagagaattaatttcgagaattttcccaatttcagatcatcggaaaattcttactataatctttcttatatcgcttctctcagttacttcaacgttctggtcagtttcggatataaatgttttaatttcttagtttccgtttcttcttcttcttcttttttttttttttctgttactgtaaacttggggataacgaattaaagcgatcaaatcgatgttcttatgattcaggatccgttatagcaaaaaaagcttgactcatg

SEQ ID NO: 41 - DNA, 1203 bp, Arabidopsis thaliana
  prAT4G21390 disease-inducible promoter
attgtatgagtatcaatatatgctttagtgccacttgctagtgcaatccacttgaggtttggctgcatcgatattagatataaatacttaagcgtcatgcaaaaaaaagattactaaataattgtagggttattatgggattttaggcgagcaaacaacatttaaactttgaaggaatacaatttgaaatatcaagatacgtaaaatatgtcacgtgcaataaaataaagagaagacccatccaaatcaagtgtgcgatactttgaccatatgagtcaatgtggcggccaaaggcaacaagcatatctaatctaagtattatataactaagcaaatattccactaaactagtatacaaagccaactgctccactaattgcctgacatacttatttattttcaaatttggtaaccacacctatagcttatacattttcttcgatggccttatacaataggaatatactattaccttctcattgttcttattatcatcaaccatttgataaatcctcatcactcttaaacattgactatgaactaaggacgtcagaagtagtccatacaagatatgaatggttggatcgtcctaagtcattgtattaatatacgtttctaatcaatggaaactatataattgtaatataatttttactaaatcatgtaacttgaaaacctaactttctttcttaataaaaattgaaccgctaaagtatctaatccatcatgtgtcaactgacaccgtccaaaatcctcttagagatgtaagaaaaagtttcaaaacaattaggtcagccaccaatcacatatttctatgcaggttgtataatcttgaaaaagaacaaaaaaagtagatgacaaaaaagaattaaaagacaataataataataccttataaagatgataataattcaaacagtttgacctttttatttcaattctctggtccaactttccaacctgacgagacaaaaatatagaaacttctcaacagcaataaaacagcaacctttcctttttgtatcccttctcctttgtcacctctcctcttacttttttatcaataggaagtttccgccattgtgacagacacagttcctctgtttctcttctttcatctttaagcaaacctcaaaaaccaatcctttattacgaagatcctcacttgtgtcttcttctccaacactaaaccccaagaaaatg

SEQ ID NO: 42 - DNA, 1203 bp, Arabidopsis thaliana
  prAT4G35110 disease-inducible promoter
ctaaaaatctcactcgtatttgtataaccgtcccacgttgttgtccacttctggaagataagtgaggtgtgtgattgattgccgtttccaacctcttttcaattaaatgcttctctctatcttcaattggtaatattggctttggtcaattcatcatcatcatcatattattattataccaaagacaatgtcacatgaaccttataatatacacaactgttataccttataaacaaaaataaaaatcaacattttattcatttttctccgctcatcacatttctctccctcttccgattctcctggtatatctctctctctctttctctttatttctctgtttgttgattgattgcatttttgtgtgaattaaaagtttggttctttgatccacaaatagattcttgatctcgatcagaatcgtgggtttgcttttgttttaagaattaattcaatgatcgatgatgaaactgattgatactgttttattgtcatatgctctgtttctttgctctgttctaaggaaattttgtgacaaacgacaatcatggaaaacgaaatttcaacctattaaaaaaatattatccacaagggtgggccttggaagtttttgtctgttcaagaaaacaagccggtcaaaataatctaccttttaactctggtctaagtcaataactgagtgcttccaccgttgaaatttagtatctcactggaaaattgtacttggaaattgcatggtcggtccatgaaagtattaaggagaagaaacaaaccaaaagtcgtctttttgtatctctctatacctatacattttatgatttgattggttttgtggggatcaatttcactattactcgtaagaaatttctttatggatatgttttggtgaatctctctctgcttctcacaatctccctctttactttttttctttcatcatataaagggttggtgaaatatgaatggaatccaatttcgttgggaggttgttgtatattataagtttttagtgtctaatctttttgaactatttgttgaaattaaggtgtaaaggtttgaccacttacttacccttaatgtgatatgttcagtcctttgacaccacaaatttgaagttcttgtgttgttctttgagtttgtcttctttgagctgattagttatcttgattttgttgcagttgttccattcatcaaaaagctatg

SEQ ID NO: 43 - DNA, 1001 bp, Arabidopsis thaliana
  prAT5G22530 disease-inducible promoter
atttcttacaaacctctaaacctttatgatgtttctagaggaccgggaaatatattgcatgcacccgtaaactacctttcaattttctacataagtcacttcaacacaacatttgatggccaaaatgtaaactgtgattagcttactccttaaagagtgaacaaaagttgcagaagtgactctatttgaaccttactctttatctgataatagagagacattaccaaagtataaaagagtttagtaatcctaacgtgtctttgctaggagttacaaaaaaaggagtcacctttgatggcttggataattaaagacttttagtagtcctttttggactaatgaaagactaaagaaacgacttttgacccaaaaaaaaaaaaaaaaaaagacttaagaaacgactcttggactacggcctaaaaatttagtacttttagtctccaccaactctttcttattatataaataattttgcctacacaattttagacaaaacttcctaaaggtataacataattgcctagaagatgacataatttttacacattttttgttgaatctgcataatgcgacacagtttaagaagaccgttaaagataacataacttcctaaacttatgcagtctacaagatgacataatttgcacattttgaaagcaaaaattggatttatggcattttagacaaaacttcctaaaggtaaaagttttacaaagtgagttgcgtggacttagtatacttttttagtttttttttttgccctctctttttggtaatttcctgcaagtgagtggtggaagaaaggaagctgcgtggagtcccaaaaattaaaattgcagaaaaacaggtcttataataatcagcttaatagctcaggtctctttttctcattctgaagattaaacatcttcaaggaacctaatctttgtctctggttttaccttcgacaaacaagcacacatattgtttgttaaaaaatcatctatcccccaagaaaaatg

SEQ ID NO: 44 - DNA, 1203 bp, Arabidopsis thaliana
  prAT5G64905 disease-inducible promoter
taaagttagatgttataaaataaaataaacaaaaaccttaaacgcagtgataatcatcctatcacaattcacaacgtgcattatgaatggtaatattatatacaaagttgacgagtgctctctatgagtaacgcatgatcgctctatgatttaataaaatatactccacgaggagcagaagcaacaccaacttcgaatataaaacacatttaacaatcttcactgtgactttagacatcatgatttttttggtgttctttttttaaaaaactcataatctggtgataagtctcgtagttacctcatcgtagaaccacaatttagtatttacacaagaaggtatggtctgattaaagtttgggggtctttgtaattaggcctacaataatcattcacaaatttgaaatattacaatttatgagggaccacaacacctaaaaaggcataaatgcattatgcccagtggcgacgaaaaaagtgcattaaaccaaaggaatatatgtttgatttttattttaatgtgggaatgggatatgcaatattacaattatgatattatctgaaattttatatctttacaaaatttgattttcaaacttaaattctatagtatgatctacatgaacactttatattcttatgcaagaaaaacaagtcctttgtccaatcattgacctcttaattgaatttgaaatatttaacaaagtacccaatagataattcttttgttcttgttaaattagattttcaaaatactaagctttagcattaatttgaataatcaaagatcttcctttgaattacctattcaacatttgtttaataacttaattcaaacaaaaaccaaagaagagcgtgaattgactttgaccaaaaccaaattctctcaagagttgctgccatattttgaccgcgcgccacgtagattagagtcaaaattgttattttattcacttcaacataaaaccaaataagcattatcggttttcaacataccggcaccaacttttccaaagtctgtatgtacctaacaaaaccggtttatcatagaaacggtcaacacaccaaaaatagttgaccaacaactacccaagtgatatccctttaaaaggagtcgcatatgtgttaccaagttccatcatcaacctaataacacacaacactaaatctctttcccaaaaaaagattaagaagtcaacgatg

SEQ ID NO: 45 - DNA, 1203 bp, Arabidopsis thaliana
  prAT1G02360 disease-inducible promoter
tttagtcacgtatttcataattatgtaaacactagtcactaatcgacagaatatgaataaatccacagtaataagaaattacaatgaatgtcagttttttagaaggattacaaatatttctgtttgaatgtcttttgtaacattatcttcttcgaaagatgagatttgtttcatagagattgtgatttttttttttttggtaaaaggttaaattttatactaattttctgattttttctgtacattgttgatacaacttattaaaaagaagaatttaaaaagtttcatagagattgtgattttttaaaaaagtttaatcatgtatttggaatggacctaaaacacataaaaacggaagacttagttgatatgatagtaaggaaccgaaatttagtttttatttttggatgagaaatcagaaaaagaaacgagtttattgtcaaaaaacatttttgtcaacttttgcacaacttaaagtgttgtaatttgtttcggctctgaaataggaacatataataaaatgtatatacaactttaccaattagttagccgccgaacgagaattttttgtgtagattccaccacattaataaaaaatacgaatgagtcttccatcaagataaaataatgagattttccatagcaagaatgagtcttcaagcattaaaaattgaaatcagtctttttcggtaatactgctacgtcgaccgaataagaatatcgtcatccactatcattttaatcgtaatacacactatctatatcattcaatgattatattgataagttgtttttattttgtatgatctcggtttcatattaagtaatgtcagctaaaaaaaatgtcattttagtgaaaagccgttatttgtttttttctctacaataaaaaataattgcttttgttgtttttttccccttcacgtattcgctttttgttgttgtcttgatatctaatctaattaaggtgtaaactatatgaaaaattataccaaaaactatagaaaaagtttcacttaacgtgaaagaagcatcgtttgatgaatgaaaacacatgagagcatgtataaacgaaccttcagaccaatgaaaaaaaagtccaccgttaaagtcaaaagtcaacgcgttgatcatttacacatctatataacaaactcatcttcttacaccaaaaccatcaagatcgatccctctctaaaaatg

SEQ ID NO: 46 - DNA, 942 bp, Arabidopsis thaliana
  prAT1G24140 disease-inducible promoter
tgtaattaaaatctgtaactaaaatgaattagtcagtcaaaaatatcataactacgaaataaatgtacactaaacactatcaaacgtcccttagtttagattttggttaataaagctatgcgtttacaggcttctaagttttaataatgttggtgcaaacgtggttgaactaattctgaaaacaatttttagcctaaatggatctaacatgaaaatctacatttttctgtttatatgtatcctaattctgtttagaaagggcttcaaacttttggcccaaaactttatatgcatgtatgatatatcaattaattttaccgatggagacttgcatgttatacccattacaggaaaaaattagatagcctaacaaacaagtcatggcgttactcgtgtaaggtggagattttacgttgattttttcaaccaataacaacaacctccggaaaaattatcacaaaaagaaaaaagaccaattcaaacattcaacgacccaaaaaaaacaaaaacactttcaaccaatttagttgattctgccgccttcaaccatttttgttgcatggttctttttccaactttgcatttaaccggtcctttaccaccggtcaatcacaatttctagtcatccaagactaaatcgggcctaatctattgcaccaagccaccaatcaactttaattatccataaaacgacactatctattggcagaagcttcattagtctttttcttcgtgagaccctttgaaacgaggacggaggaagtttcctttgaataatgcagcattttcaacatagaaaattctccaaacgcaacgcagaaatgacctctgctttctccaacgtcaaacttgtttagttttatctatgtcatctctctaattctcttgtaaatctcaataatacctcttcacttaatctttttaatatttctcttttggatg

SEQ ID NO: 47 - DNA, 667 bp, Arabidopsis thaliana
  prAT1G24145 disease-inducible promoter
gggggtactgtttgctaattttggaatatttttgctaatttcatgattttttttgttaattatgggacttaataataatttaaaaaaacatagaaaacgtaaaaatttgagacccaatataaatgtttcaacattatgcgtaaaaggacaggccttcctaaaattagaacattttatatgatatcctcttaagtcttaagtactatcgaagccggagatttctaagttttacttttactaattaagctattgacacttgataaatgccatatttttgcattccctaaaacctcaaaactctctaaacacgtttttgtggacatattaaaatagtgtattttgtagtctcttccaggtttcttccttcttgtcaacgtcgcacaagtccgtttacggatatgggtgaataattgaatattatccatgtttccttattagcagtcgctattgtgggtttctttttggtagtctaatttattatttacttctttaaaatggggaaacagatacaaagttgcattacatatataacgcaatacaattagatcccatttgaccgagtaaacgctaagatccacacaaactcaatcgtcaactcctctctcttctctattctatatagttgcgtgaaatgtaatgaaagtgatcatcattcacttctaagatg

SEQ ID NO: 48 - DNA, 1203 bp, Arabidopsis thaliana
  prAT1G35230 disease-inducible promoter
cttctaagaatagttgcaagcctttaaatactccgacaaatctggcattagccgaaagatattccaaactcaaaatcggatcagatagtgtggtggtctaattttacctggatcgggagatgtccactctgtaccaccttgatgcatttttactgatactgatcagatcaaccgatataatatatatataaaaaaagaaagttcgtccaaaaggaatcattattttcttaaccaatagaatataggaaataataggataaatctatattagtggacaggtaatagaatgctttcattcacattgaaatcatattgtaataagcacacttttcttatcaaaaaaaaaaaggcaaaaagaaatggccacgcaataaaatcattagggtaagttgaattttggtccataatattataaattaatttaatctcgaaagcttaatcttatgatctcatgtgatctttattgaatttacttacttccatagagttttgtattttgtctaaggaaagaaaaaaaaagtctgccagctttggaacgccgcccattcctctagactttcttggaaacaacgcgttgttcttgttggggtcgacgaagactcactaaatccatccgacgactcagattttatcttggcttcttttgatgtgtacacatatccaccctgatttgattcccaaagccaaaagcctgaacaatgtagtgtagaagaagtgacgggaaaaaacggtaatgaatccacaatggatatttacagaaagaaataaaattatatagattatagagaagcaaaattatgcaaataatctttatttaatactattaaaagagtagctgttggaaactataacaggtaatttaaaatattttacaagttcaacatataataattttgaaattcagtccaacataactatcagtatggaaataagccaaacaaattactcaaaataagaaatatgttttcacattattatttaaacatttttagtcatttgtttggcttatttccaaaacgatatttatgttggacttgttttcaaaagtattatgttgaacttgtaaaaaaaatttatatagctgttgaaatttccaagaaaataaggttttacacctaaacccttccactatatatataaaccccacttttgtctctatatctttactaatttcttaaaccctctcaacaatacgtaacaatg

SEQ ID NO: 49 - DNA, 1203 bp, Arabidopsis thaliana
  prAT1G57630 disease-inducible promoter
aagcagagatgaggattgtgagtgaaaaaacaaactcgagagtcgcatggtacagtataatgtcgctggggatttgcattgtggtctctggtttacagattttgtacttgaagcaatactttgaaaagaagaagcttatttagatcatggatagtttcttgttgaagattactagaaccaacagtttgctctgctttctttctcttgatcttctctttggaactgttagtgtaaaattttgttcactcatttaacttgtaatttgtctcctattattacatataacatataactagactctaattataggaattgcaactaaatttctcttgataaaaataataatgaatttctttggcatgtgtttttttatattatctgaactgaaccaaattgcagaattgtgacttgagaaaaacagaggactctgttgatatgaagtctcatagtataaacagaggactctatgtgaaacagagttacgaaaaattctaaagtaatgaaaaaaacacattgtggttttatctggattagaaaagccttcatatattttatctgacaacattaagaagcttaggagatttcttgggatgcaagcattttgtaacctgttttttgctacataacatgacattgtgttccagctaggtcaaaagagtattacagcattattcacataacacacagagcacaaagttaaatctttgtagagtttctataagacgacaagaacatatgtccacaatacgaaacataggctgatgcatgtggcgggggcaactgtggtggagaggttggtcataaatttggggcgtacacggaaactcagacttttcctcaatacaaaacatagtctgatgcattacgtcgtcggtgcatgattgtcaaggtagacgccgcaaataagcttctcggaaaaactctatacatgtgaacgtagactagtctatggaatccttttttgctatgactaagtccaaacattgaacttattgcttggaaactgcatatatttgaatttagattttgatggagactttcgttttcttcgctgaaattgcttgtgcgtttcattgctatttccttcttagaactagagaaccacaaccattttctgagttattacttcctacatagctctataacctctattattactttattagatctctgatcttaaatcgtctctctgtcgtagtaatg

SEQ ID NO: 50 - DNA, 1203 bp, Arabidopsis thaliana
  prAT1G67810 disease-inducible promoter
agtgggaagtatacgaaacgaattatttaagatatttaatggaaaaaagttttagaaacaagtaaaaaattggttttggtcagtacactcaagtgtagccgtaagctatgacgacgaaatgaaaacaactttaaattatttcccaagcctctttccgacagtatggggctctaaaagtatatcactaattcagtagtcgaatcgaatatgcctttgttctttcccttttaaatccataatttatttctaaaataataaaaaggagattgtcactggagaagccgcctcaaatgatgtccatatcgcatcataacatttaacgtcacacgataaaacaaaacatttcgtattttgttttgcaatctatttagtaattgcattattagtagtggcctatctcttttgaagttgaaagtctctagttgacttggtcgtatttgtatcattgtcacatataagtgaaaattaagatggacatttccgatattattagctaaattaaatcattctaattttttttaccagatattcaaaattccaactcatttagagtttcacgttggcatccaagtacttggattagctttcgactaaacaagaaattttacgtttttgcatagattataaaattagtttgaaaatttggttcaaaaatttcctgattaaaagaatataacgaagcattttataatgatgacaatgattcataatttttgtaagtgcttcagttgctacctaactcaaagtcgcaacttatattctaacagtttcatcattttaattttctaaaaaaaaaaaaaaatcaaagaccaaaaaattctaaattaaacctataatagtttcctttaaccaaaaaaaaaaaacctatactagtttcaaggctcgtagaggaagttagattacttaccatttaaaaatctacaatttaaatatataatttttttaaagtattttataaaataacattacaagatagatctttttttttttggtaaaaaataagatagattatatatagatagatcaataaacataaaaatagttttggcgcgttatagaaaggctctatgcatctaatcactatcctctctccgtcgcgtagttcaaggagttctcctttgattatccttaaatatctctcttctctcgttttcttctacctcctcctcccatttcttttcattgcccttgttacggaacatg

SEQ ID NO: 51 - DNA, 1203 bp, Arabidopsis thaliana
  prAT4G18250 disease-inducible promoter
tcaatacaaacacaaaggacaatttggccgagtggtctaaggcgccagatttaggctctggtccgaaagggcgtgggttcaaatcccacagttgtcatttagatttattttttccagactttttataagatttagagactaaaaaactttttaaaataagtataacatagattgttgcctaaacgaatattgttacgtaactgtaaggtttaactttgattttggtccacaacagaagagcttaagccttttacccatgtttgatcagttttggatgctaagatatagagagtgtgcatagtcttcactcttcactcttcaaattgataatgttatgtttcagtagaacgatcaacgcaaatagtgaaagcctaatgagtggccaatcatcaaaattagagcgaataaaaaggaacaaaaaaaaagaagacaatgagtttaatactttttagcctcaacaagtttcaatcttattttttttgtaagtatctagctagttctataaaatttatttaacttagtgattagataaaagggaacaaaaaaaaaaatgactttggtgtttgctagcctctacctcaagtcctcaacccatcattttatttgtgaattaggtagataaaaatttaaaaacggacacctaaaaaatctaaattctaaatctgaaattttaaactcttttaatttttttctagtaaaatgttacgaaagtaaatcgtttgtcccataactgttaataaatggtaaaatcattcgataaaacagtcgttttttttctcttttttcatgaaaaaacttattcccattgccactaaatggaattatttagaaaaaaataaaatacctcttctttcttggcaagttcccggaaaaaaatatggattgggaaaaggatagtattgtagttacatttatttcctctatctgtttccttccttctatttattcttgattgactctcatcaccaacgaatatgtactagtaatttctactaacaagcgaggaagaagcaagagaaattataacgcgaaattcacttctttggaacgtatatttttttttctttcagcagccaatggcgaaaaggctgccattgattttcctccttacttcacattttttagtatcgggtaggattttcttttcttgattgataataacaatggttttttgttaattgtttcttatttctaagcttaatg

SEQ ID NO: 52 - DNA, 1203 bp, Arabidopsis thaliana
  prAT4G35180 disease-inducible promoter
tattttaatgttttagctggtcatcatcaactcgatttaagaaaactgaattcataaatttttagttgactacttgactggtgttcgttgactagttcacttatttaagtttttcttattaccagacctagaaagaaaaaaatgaaaaaaaaatcaccaaatgtccaaatgagatagcaatgtagtactagttaatagattgtttcttaagtcttatagatttgtatcaaagggcttcttgactgatacaagtttcttcctttaattcggtttataatagtattgatctaaaaccaaactacaaatacttacttaccactaaacttacttttcaaactttggcttgaagctaaacttgaagctaattctcacttaccactagacttgaagctacttttgaaactttggtctcacataactagagttgtaaaattagatgactagatgcctactctatatactttttaattatttttgctcataacaaaagtcgagtcaaaaacaactcgcacgtttatcatctttaattaactacttgattaatacttattaatcgtgtatacaaagagacgatgaaggaagatcagctcaaagttgacccttgcgttgaccaaaacatccgaagagcaaacaaagtccaattgaacaatgaacacaatctaacacaaactagtttggtttactttttagcctggcttgagttttaagctaccgaacaaaattagaagacttcgatttataccgatgggtctcgctttcgagagtatttgaaagtgacataaccgattacgtcatctttctcgtgtcattaatgcttacgtcatagctaataatttctaccgttcagaatatattttcttatatggtaattagagatatgaattgtttagtgttaaagtattgagattctcttgagcacttaaacagaaaaaaacaattccctaagaaaaataccttccttttttttgttttggaaaaagagattcaaagtcaatatacacagccaccgaacaaattactctatataaatccaatgaaagcagagtaaacatttatatagccatacaatttgtggctcgacgtaaataacgcgattggagtcgttagaggaaaataaagtttattttgtatacaatgtctatagcattgggaaacttatttgatttggaatcacaagaaagtggtggttctcctttatttatg

SEQ ID NO: 53 - DNA, 1203 bp, Arabidopsis thaliana
  prAT5G18470 disease-inducible promoter
aatcagaaatctttgatcccactgacaacgtgaaaacatttttactgtattggttggctaaatgagttttagttattacaccaagagatttgagttcaaacttcaaaatatactatttttgtttgagtttgttgttcaagtattacatagctgattacagaaaccaaagaaatataagatccaatgtacttggacttcttttctttttttgtcgtcaatttcattaaatccttaaaaggattacatggtgaaaggaaaacccacatatacacaaagttaaagcccaattaacccaaatattgattacgatgaaagaacaaaatgggctaaaaagagacacgtgtgtacacgtggaggcatggatgaaatataagatccaatgtacttggacttctaaaataacaaataatgtaacactagattttagaaaacaaaaaccatccaactcgatcattcatcaatattatatagagaaaacagtcataatgttagagagagacgtatgaatcgaaattcatattactgtatttcgattacctaaaaagtttcgaaaaagaattaaaagtttgtgtggaagattccaatacacgttgctatcggaagaagacttggaaaattattcctcaagtcgttaatgcttggaattagtgactaacaatcatcattattccattgaaagagccgcccacgacgcgtttccattttccacaccagtcaaagttgatgtctcaaattagtaactgatttacaattttaaattacaaaatacaaagcccgtacaagctaatgtaaacggaagtaaaaccaaatacattaacaacttttagtttagctgttcaaaggacagagatatgacctcaaccttaacccaatctttcgtctgtgtttgaaacttctgctcgtaaagagactctagtaaaacccttcggaagaaattttttaggtagtcgccatcatataatttaaagcttatgagacttgttatgttacaagcagagaccaaagactattcataatcacttatcagagtataaaatactctttttttgtcaatcgagtataaaatactctgatatcacttatacaaagttctacgaaactcattaaagatttgagagtgtacaagggacaaaaggtgctataaatatctcttaaaagagagctaaaagttaaggaaacacttaaccaagcaaacaacaaatg

SEQ ID NO: 54 - DNA, 1203 bp, Arabidopsis thaliana
  prAT5G48540 disease-inducible promoter
aaaatataattattcttttataacgatggtattattttttgcaaaaatagaatcatataaagatgagaggtgaactataataattaataaaaaattaatatgataatttagataatgtattttgttttaattaaatttaattaattaaattagtatttgactttttaatttttaaagagatgaattaatttactctttaaattttatttctaatggcatacctatgtaattacttacaaaaaataagattatatttaaaatgtacttcccaaataatatagcaggaagttttcatttttaattatatatgtgccttttcatttaacaaattttccaacatgaaaagaagatatgttggtaaatggtaatctacaaacccaaataaaaaagaatataaaaaaaattttagttaaagataaatataaattctttatcaatactatttccataccaacttgtaaatcttcaaattattttgattagtatctatctacaccaagtaccggaacaagtttttgtgcaatacggaatcttcacctcaaaccaagcctaacataggcattcatcaaggctgaaattgtgcacaataacacgtacaaaaaatcatcgtaaaataaatacgaacatctctcgaaacagagaagtggtcaatggtggttatattttttctttttttgtggtggtgatattatgtaatatgattagtcattgaagaccttttgtagagagaagtggtcgatgttgcgccactttagcgttaacgttgacgtttggaaataagaagcaataaataaatcgacggtcaataattcctgctccatatatgttgacctacacgtgatgccattttggaatcttccaacgaaaattatttagttttaattcaattcaatatattatccattttgtctttctacaatacacaaatgaaaaaaacaaagtaaaattgaccaactaatatttatttactgatttgattggtcacaaactcacaagattttggccacaatatagacttctcggtcaacaaaaatttgtatttgatcataaataaataaacaattatttcccacttgttgttatgcgttttgaccgacttctaaaaaaatcaacgttctagaatagataacgttttggtataaaatcagtctcttcttctagtgactcaaaacaaactgtaaagtttattaagaaataataatg

SEQ ID NO: 55 - DNA, 1203 bp, Arabidopsis thaliana
  prAT1G30700 disease-inducible promoter
caatatgaaagtcaaaccagccgctcaaatgtatccttcatgagttcatgttatatttcagtcaatacttttcatatttaaacacttattataattacgtaatatttttttgcccaaaaaaaaaaattacgtaatattcaacatctctaccttgtagagttccaaaacattgtcacaaaatatttataaagaatttattttaactaattaggtcgttaattgtccaagggtttttcatagttgatatagttctgttcaaatatagccatccttaatcgattcatgggatcgtaaattactacttcgagtgttgtaaaaaaaaatgaaacttctacattacaaactcgaatttaatgcatctggagtgatactataaaagtagggatgctctcaggtcgcatttgagagacacagaaatgattttaatggaattaatatattttcagtttttcacaaaaaaaaattgtgtttataacaactgcagattcaatgctgattttatgagtctcacctatagaatttatatttctatattcatagaggcagtataggtgttgacccaacatcgaaagaacacttcgtaaaaaattctttggaacaaggctgaaaatttactcccaaatttagctatccgatgaagataaatcatttaccgtttattaaagaattatcgagattttagtccaaaccaaaagagattatgagcctaagattttgaatttgtattggtaaaagaaattgaacgaaaatttcagaaaaaaatattaataaattgaacgatagagttcacttactacatagtcaactagtgcctagctataatagtttcaaaagacaaaaaaaaacaaaatcggttaactacttccgtgacataattctcattttgatttttgaatccagtctaatttgaaaagtatattcaaaatctttaaatccattaatgataacttttataatacgttgacacacgcaattgtatatacaatattcttgaattttaaatgtaaattctagaatatattgcgatcaccacactaatcaaaatctttgggacaacttgaacccacatttgacttttcttggtcaaatattttggcatcatgcatgatcttctctataaaaaccaaaaggcctcaacgacattcataaactcagtcattatatttatttttgttgtatttcaacgttcaatctctgaaaatg

SEQ ID NO: 56 - DNA, 878 bp, Arabidopsis thaliana
  prAT2G29460 disease-inducible promoter
tctcataacatatttttgttctgtaatttagatgataaattgataaccaatttgtattattacgaagaaaaagaaaacagtttgaactattcattaaaaaaagttattgtttaaaaggtattttattagattaaaatattaaattaattgtaattcacttttggaccacgcatttagcatcacacgtataactttaacaaatcaggtaaaaccaaatttctttaattaggtaaagaaacagaagactgaaagaaaaactagtttggaaacaaaaaatggtatttgcaggaattgtaaggaatttgggatactagaaatgtatggatctaagttagaaaaaatcaacaaaaatttgttgaatattatttataaagttggtatgtttgaggaaattgaaaaagataaaatatatttgaatatatggagagatgatataatgtgttcacggttcattggatatttagcagtgaatgaatcaagagaaagcaaaatagttattttcttctttcccgtcgttttagtatacttttcaagcacgagaacggaatcacaaaaactagtcaaaaaaggcgttaaatcctatagaacaaaaacatataagctatggtttcgaacgggaaccaaaccataatatgcgatgcacttctaatagcaatcaaaaatttgttaatatgtacatatatatttttgttttattaaaaccgtatcatactttatctgcagttaaaccgcacatctttattcggagcctatatatctacactgcctatatatcaaccgaccaaacattcacgaaaacaaaccacataaactagtcaaagaagaatagatgagttacattataaaaaagttcaagtgagagaaagagaggtccaatg

SEQ ID NO: 57 - DNA, 655 bp, Arabidopsis thaliana
  prAT2G43620 disease-inducible promoter
accagggttggtaagactaaaccgctttttattgatatgctggtttaattttgacgcatgactatttggaaattgcaataattgagttggatttttctaattttggttgattttgatttataaatagaaacattttggcttcactagtcatttttctcacaattccatacaatttttgttaaaaatcaaagtaagactttaaaagaacgttctaaatgctatattagttgaccaaaaaaaatgctatattagctaacaatatcgtttgagctaattaacaaaaacttggaactattcaatagaaaaatctcaaacgtttgaactaatctaaacttgattatctcaatcaagtttttatgagaatgattttcatccaagtaacttggctctttaaaattttgattacatattcgtttttgatctgatctatgaccgacatggaatttctcataacgacaagagaaaaaactgtgtcattgacttttgttaagtggtacaaagtggcattgactttgactcagaaaaagccaatcaataatcgtgaaagatgtctaacactgatcaatatttcaatttgaatagaccaaatttacactataaatacatcaacacaccttcttcatttcttcacacaacaccctccatacacaaaatg

SEQ ID NO: 58 - DNA, 1203 bp, Arabidopsis thaliana
  prAT3G02840 disease-inducible promoter
ctacgatgattgaaccaactggcttatgttttacctatctgcgttaaatttcgtggtaaactgaaccgggatgtaatgaatccggttttggtttgattttttttaaatgaaaataactttctgtggatcttttaatcgaaaataatattgaacgttacataattgggtgaaaattcgtatttatcagatatttggttttgatcgaatcttcgtgtggtttatcttagatactcgaacttttaaagcagtgtatgaatctacgcgtaaaatatttaatgtattgaagtaatattggaaaaacaatgacgcggggaaaagtaaacaaacacggacccgacgacggataagcgaccaagcccgagtcgcatcgtcttggtcttaagtctttccctactcgttttatcattttcttaaactaaaaactaatttaatcactcttaattatttacttttttcttttaattcaactatgactaattcttattcacagaatgagtctcacatgcctactcgtcgtactcgactccagtccaaatgttcccatacttgtaatctatactacatatattatacgtcatcgtcatgtatatacaagactcaatatataacgtccaaattaatcaaagatttaacatgatcgggaaccaatatctaatcatcgattagatatatttaagtctagtaactaattagtatattttctacaaattgtttaattaaacatatagtatacagttggtaaaggggaaacacgcgtaatctatatgtatatattaataatatatagttcttggtatgatcttcttagttatttatttactaagctttctagactttaacaatatttgaatgaaaaaaaaatttaattaaccgtcgaaaaaaaagtcataaatctggacgcagaatactaatccacagatttcaacggtcaacattccaattcagttcacaaacctaaatcaaacgttccatttcataccttttttctctttcaagcttatacaacctttgaccatgtctcactttattaataagtacaaaaccaccttacattacatgcatatatataaataaaacacgttaaggaacatatatttatacaaatcccataaaccccatttcattcttcatcgaatagtcgaaaaatatttgaactttcttaggaaaccaaaccaacaaaacaaaaaggaaaacgagataatg

SEQ ID NO: 59 - DNA, 1203 bp, Arabidopsis thaliana
  prAT3G26830 disease-inducible promoter
accacaaaaggtgtgttttataccctatcatgttttactcttgagatatgttctttgacaccacccacaaaatatctctacgaaatacgaagccactatatgtctctttaatttcactttttatagtttttttcaccgctaaaattgttgactaaaaaatatattgcataaaaataattgataatatatttataagaaaaactatgataagaaaaaatataatcggttgaaatgagtcatgactaacaataattaaaggttaggaaattaaaagaaaataaattctgaaataactaaaaaaaaaaaaatagaagatgatgatatatggatccctatactaatattttggaagtacattgaaaaactaactttcaaagacccaattaattaagctcattaaggataaacatgttaaatactaacttatggacattaattaaattaaaaattataaaacgaaaataaattgatgacaaaaaaaaaactatgaattttcttattaggatttgtaatctactttcttgaaaaaaattgaagtttactgacggcttcctttttttggaaactccaaaataacaaaacatatgaagaagttttggaatagcctttgactcaacaactttaacaatagaaagaaaacatgtttaattaatgttcatgcacttcgtctcggctgccccttgtggcctgtggggttgccgggttggcttagcttgagacgacccaatactgaatttgttagctcggtcagtgaagtctacatgcatgatacaaaaagattgactagtgtttaagttttttttttttttttttttttttttcataaatggtagtgtctcatattagaaatggtagtttgaaaagtattcagtttgtttgttcactttggattatttgattttggttttgttaattgaatcagttttgtttgaaaagtattcacttttgaaaagtgttcactttaaaaagttttgtttgaaaagtgttcactttgaaaagtattctttgagaagtgctcagttttggtttgtccacttacgattattattcacaagctacagcggatagtagtgactagtgacttatgactttgaataaagaatttccctctaaaggaatgaatacattataaatagattattaacctaagcttgatagagaagacagaacaaaaaaaaacacaagaacagggcaaggaaaatg

SEQ ID NO: 60 - DNA, 1203 bp, Arabidopsis thaliana
  prAT5G12930 disease-inducible promoter
tgattattcaaaagacaactcatgtgcccgcgcatgtgcttgacgttcgtttgccgtgtattagaactttaaaaactgattcatataaatcatcggtcgattgaaatatattaacatggattatgatgacaacaatcattatacaattaagttttatgaaatgatcatccacaaaacataacttttgtcggctatttttttgttgacgtgacaaactaaattagtaaactgtgttttgagtgcaaaaagatgagaaatttgtgggattgattgcatagaataacacttacaaaaatgtaggtagtatgcaggggagtcaacaacagacgacttttcataattcagaaaacgaagaaaaacaaaaagaaaggtcgatttcccaaggactagggcatagcacgtgtctatatcactggaggatatatcacttagaccgttagacgtcacgtgtaatcgtgtattaatgcttgcaaaatgtggttcaatcaaatacccaattaagggttatgagaactaatacaaaaatgtggttcccgtaaataatgcatgagcaagcacatgactgacatgagagatagaccagcgaataaaggttaagtattgacgcaccatgcaaagcgtaacggtgaactggagctctactggctgagataattcacaaggtgaaggtgagatatattttaggagagccagaaaggtagaacccaagacaaataaagagagagaccaagtgagttttggactaatgtttttcaaagaatgtgtctataactatttattaagttccaaaaaaagacaaataaaagtatgatttttctatataggactactcgattaatcttaacaaaaagacgaataagcagaaaacatatatgtttgttttttcaaaacaataaccttggaaatcaaaacaccagaaaaatgtggctgtgaagaaaagtacaagagagacagtaaaaagaaaatgaaccaaaaaggcaaaaaaatgaatacgtggcttcaatgtagacccagacgatgaacgtcgcatcaccttccttacgggggaaatctcacattttgaaattacagaaaactccaaagaacccaaaattgaatattgaatattgaaaattgaaaaaatctctctttgaagaattgaaatctctctttgaagacgaacgaacctcgttgattcctccattcctcttcggctcgtccatg

SEQ ID NO: 61 - DNA, 1203 bp, Arabidopsis thaliana
  prAT5G24110 disease-inducible promoter
taacagacatcaccgaaaaagtcgccgaaaaattgaccggaaccacatatgtaaaaatgagttccaacaagaggcagtacaaaagaataggcataaccgtaccgcataagtgttcaagcccaaggcaaagccatatctaaaaatattttcatacaaaaaggtgtatacctaaaattttcttttgaagtaagaacaaaaaaaaagttaaaatttattttcatatgttctcgaaaagtatatttattctaatatgaaatggcaaataattttgccccgctggaatttctatgtagtttgcgtttattagagtttgcgtttgtatttagcattattattatttgcattgtcgttagcttttacatctgcatttagtgttatgtttctagcttttgctttggcatttctattttttttttgttagagtttgattttgaatctattttaagatttatatatgacgaatttatttattatatatattatgaaatttcatatgttatactatatttttctaatgtatattgtttagtctcataaactttttaaacgcctagattgtctaaacgtcgattatgggttatatattgattattgacactaggtgatcaattgtcactccttattgtctacagttatcttaaacacttttatagaaactctagaaatgctatcttgtattgaatttggttccataaatggatcgataattaaacttgatccaaaacaaaatgaatattccaacataatgaccatatttgtctcttggaatttctaaactcaatattagaattttatagcaaaattaaaatttacaatagaaaatcatatcctaataagaatgaatagttattaacaaattaatattacgaatgtaagttaaatgattgtgaccctaatattaaaacaaaaccagatcatgtaattcaaaatccaataaatcaaaaataaaaaggatcgagaagcagagaactggtcagcatgttggactttccaaattcattgaccaaagactggtctcacttctcacaaaccacatcagctttcttcgttcttcagtcaaaaagtcaaactatctctctcacacatcctctttaaattctcctctttctcagtttccagaagccatgcaaaaataaacatagtaacaatactttaaactatttacaccactttaatcttattctccactctttgaacgtaatg

SEQ ID NO: 62 - DNA, 1205 bp, Arabidopsis thaliana
  prAT2G18690 disease-inducible promoter
ttgaacatgttcgggtttgaaaaccggtttatgtaactatcaagaaataatcaaaacaaaaaatccaaaaataaacttcaatgtgaattttgatttgggctatatatttcaaatttcagtctaaaatttattggcaacaatattattaaaaaaaaaaacctacattgttattacaatggggaaacttatttattaagcggtataaaaggttttatacataagtaacgaaaaagtagtaagtaaacgaccatgagttggtaattaatttgcaaaaatgatggatgatacgtaaaagtttaattatattatgttaaaaattttacttaccttaaaaacaaaagagatcagatttaaagaaaagagaggttagattaaaggaggaagttctacaagaaggagaactatggatatgttttggtcatggaagaaatagtttgctagatttgtaagtgctctgtttttctttttttcaatcatatttggattagaagttataatcatttagcaatttgcaacgtcctcaaagaatgtttgaagctctaattcttctatgttcggttaaatataaatatataatgacggtttggtatagcatatagtttgaccctactgtttatgactcattcatgatgacttgttcgcttgaggaaacaaattttttttttagatagatgtttcattatcttacacggtttcttgacattttcaacacgaatagaggaagtgaatgttgatcacgtacatttgatgaagaagtcaacttgcaatgttaaacatgttcctcttattttctttatataacatcttcatttatgacgcggtcaggaagtcatcatttttagacttttcatcgatgcattatgcttcgggttctttgttcttggatttgtctaagtcttggtttcttcctttgattttggatataatatgtagtaataataatatattgagggttccgtgtacttggactcaatcaaatggatgacaagaatctccgtaagcgaagctaccccgaaatacaagccaaagacttctttttccatagaaaaatagctggtcataatctaaaggtatagtagtttttgaatgtttggttagtagtatataagccctagacagataggtttgatcttataaaccctcgccaccattaccaaaaaccaataagccaagagcttttctcatttttcttcttgaaacccatg

SEQ ID NO: 63 - DNA, 1205 bp, Arabidopsis thaliana
  prAT3G22060 disease-inducible promoter
gatcaatacaaaaacattaatttctagaatatttttgtcaataaagtaccataaatgtaaaccaagctaaatgggttggtccacttgcagttagatgattcttgcttatttacataaatatcgtcaaaaaatttagttgtacgcttattgtttgattatttagatattttgagataatgtaataagggacgtatctactatctacgcagaaaaaaacagcattgcagctgcccatatgcgtgaaatgaggctggccattgcaatgcattacttaggttgatagtaaatttagaatgagtgatgaaacatttcaacttattacactaaaaactggaaaaatgccaaaagctccatatgcgggaaatgaggccatcatcaccattggcttgtaccatgactccaaaacgcgaggacttaaagcttcgatatctatctaaactatttatgaatggttcacagaattcataacccgcatttggtctggaagattagtgattcataatcctgattattataaaagaaatttcaattaatatcttaagatagtttgatatccggcctatatatttttcaaaaatgttatattattcattgaatatttaagagtggatattttattttggggctctggaggattcgttccaattaactcgaagattttagtgtctagctagctagttaggcctattgaaagctacgtgtatagaaaactcacattcttagacttttcaaagcataggtttagagagatattcatgaacggtgatttaatgtataacattccaaaactatgatgatatgacgcgatgactttgtttcattcattgacttgaatccctccattccttctataaattagtgcaaaatgctacgattttagtatataaagttgcaagcttaacattaatcatgaagatgtgtaatggatccagtttcttagcctcattaccactgttattgcttcttctcagcttcatattggcttccttcttcgacacggcaggttcaatcttcttttaacctattgattataccacattggtctctttctgattcgttttcagaattttattcttttcactaatgattcttcttttgaattttaagttggacaaatcggagtgtgctacgggagaaatggaaacaacctgcgacccgcgtccgaagtcgtggcgctttaccaacaacggaacatccggcggatg

SEQ ID NO: 64 - DNA, 1205 bp, Arabidopsis thaliana; identical to SEQ ID NO: 63
  prAT3G57240 disease-inducible promoter

SEQ ID NO: 65 - DNA, 1205 bp, Arabidopsis thaliana
  prAT2G18660 disease-inducible promoter
tttttttcttaacaatatacgttttgtaaatttaaacttggacgatgatataacaacacaaatgacatatgcttaaaagttaaaatctcattttttatattttgaatctttgattgatgaattatagcgacaagccgacaaaggcaacttcctcggtaagggcaatcgttatcttaagtttaatttgatcaaatctctttctcagcgatagaaagtttaattggtatatagagatttgggcctttacataaaatgatatttgaaggcccactaagcccaattattttccagaatgttgaattcataaacgcagatttacttgacatgataacaaagagaaatttgtcttgattcaaaaaaataaaataaagaagagacatttgtctttctcttgtaaaagaggtcaataaagcaaatttgtttttcatacttcatcatttgactaattttattggtgttatgtaacaaaccgaatattggagatatcttagggagcaagtacgtgaagtccgaagaatattctagatttcactattaccttttgttcaagttatttttttatatgtttagaaaagttgaagaacaatctgactcggataccatgatagatttgggcttttaatatgagatttcaactaaaaatcaattggtaataggtggagtgaccctaacactttatatactatttgataatttttaatttttaatgtgggactttcttcattaacacattttgttttagtagatggtcctaacgttagaacctaacactcatcagaaggtttaaaagacggttatacttttcccgatggttttggatttgggtaaggttgagaattttctcaaggtaggattcgaagtgatgttgagaataccctccttagtttcttcgaaatttccttcgcagctttgtgaaaataatatccacaaagaaaaaaaaaatgaactttaaatttcaaaccctcgtggaattttccttcacacatcatcatatattcatattcattcaattcaccaagaaaatttaggtggagtaaggaataacaaattgtcctgtatgaaatcaatacaataagtaatggaagacttgacgtagaccaaagacttttccttttacttacagtctttgagtccaattatatataaatactcgcttccctttgcttcgttatttcacaaacaagttaaagaaaatgataaaaatg

SEQ ID NO: 66 - DNA, 766 bp, Arabidopsis thaliana
  Drought-inducible promoter fragment from prAT1G16850, in GenBank NC_003070
attgggtacgattttcataggtctttcctcacgccagaagtgttgttttattttgttgattgagttattaattattggaagcttttctttcaagcaaagtaaaatgcgtaataatgattagtcacatccaatggttagtcagtctattacaccgttaatcaagctctggtcatataatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatag

SEQ ID NO: 67 - DNA, 604 bp, Arabidopsis thaliana
  Drought-inducible promoter fragment from prAt5g52300 in Genbank AB019226, GI3869065
tgatgatgatgatgaagaagagaacgaattttgaaattggcggttttgaatttttaagaaattaaaaaatatcccccgtcgatttcaagagggagatggagataccaaagcaactctcgcacttgtcgtcttttaattttaattgagtacgttatgccgttttaaatgttcaaaacagcacacagttgatagctgaattgattttttcttttgccgttttgttatatttaaacaacacacagtgcatttgccaaataactacatgatgggccaataaacgtggaccgactaaaactaaataatagaagatacatcgataggcttctctaaagatcggataaaagataatgtcgcatagccacgtagagagcaactggctgagacgtggcaggacgaaacggacgcatcgtacgtgtcagaatcctacagaagtaaagagacagaagccagagagaggtggttcggccatatgtcatcgttctctctataaactttatggaactttgttctgattttctcagagacacgaaaagaaagaaaacaacactagaacaaagagggtttgattgattcacttgaaaaagagaaaacacagctttggaaa

SEQ ID NO: 68 - DNA, 907 bp, Arabidopsis thaliana
  Drought-inducible promoter fragment from prAt3g46230 in Genbank AL355775, GI7798991
ttatttattctcaattttcccatacgaattttttgtctttatatttatcacaaaaaaagagtttgctctttaaaaaactatactaatgtaatttttttattttattttctctatcttaatcggatattaatccgactcttcttcttcccaaaaattaatattagtttcaaatccaaagcaacccacctcattcaactttccttcgattttcttcaaatttccagtttccacttgctttcattgcttctttcccgccgtttctagatcttcaatcgagaaagggatttgcaacttttcacacaaaaatcttagattaattgttattaataacttgttcatcaaaccactaaaaatcccgtgtcatcttcgacttcttggttaaaattcaataaagagtgtaacttttcattgctataacttaataatttgtttgtgagaagagaactctagtcttacagggaccaacaccaacaatcaaaatttagataatgaagaatagttgctgatgcatgattaagattgaatttatcaacaaaagataagtgttcattatacaacacgtgattaattgcatggtgtattaaggcccattaacgaagtccatggtaaaatgaaacggcatggcgttcactaccccacctaatgaactgcatgtcgtctcaaccatcaacatagaagcttcttgaagccacctgagaaatctggtagcgacactcttgaaagacacgttataaagaaacggaaagaagaaacctgaaatttcaagaaacttgcagagctttctatctcttatcctcttctctaccatcatttctccctataaatacgccaacgcacataagtgtttgcattcgaagagagttctagcaaaacaaaacaaaacagagcaaacagagtaagcgaaacg

SEQ ID NO: 69 - DNA, 780 bp, Arabidopsis thaliana
  Drought-inducible promoter fragment from prAT1G52690 in Genbank AC008016
agaaagtgtatattttagtaaaatcctaaatctaagcattacactaacacgtggaaaataacataccattgacgattgacatggctaattttttgtggaggtgaatagtttgaggatttattaccctaacgttgcttggtcaagaagtgaagtaggatgacaggcaataggaagatcttaaacctttttttccggtgacaattatttatgactttttattgttgtcaaaaaatatattatcagtaatatatcaataacgaatacaataaaaactcatccgatcgattttcaagaatttatagctatattaaaattacttcgaatccatgtaagaattgtgtattggttctttttagaaaaaagtaaatatctatgcagtaatggcggttgcataatatatgccttgagtagatgaatatccaatatcaagataacgtgagtcaccacgtgtctaacatcttccgtagctccgtttttaccatgacgtgtcacatagatataggtcatcatgaaaacgagaaacctaactttaacactcgcacataactccaagtttcgaaacttcgtcacatcaacctaatcggggcacgtacctacacacctgtcgcgaaactgcaacacctatcttgttctctcgccgaccaagacttgctataaataactctgactaacgagtcggagacaactcacagttccaaacacacaaaaaacacaagatctaaaaaaaaaagcttttatcatttagaaaaatttggtttcgaatttcttcgaagagtgaaa

SEQ ID NO: 70 - DNA, 447 bp, Arabidopsis thaliana
  Drought-inducible promoter fragment from prAt2g37870 in Genbank AC007661, GI6598780
aaaccatatgttgttgtagcctactcatttctatctgttttactacatttccgttgttatatctaataataagaattttcagctcgaatgttgaatccttatagtgtctatattgaaacaatgaaaaccaaaagtgttctgaaacaaagagagtgcaaaaagttgttggagcctgttttatgaaagaaaagtaaagagagaaacaaaaacaaacacgcaagaaatcaaacgactaaacacacaacagatggtgaaatctaaatcaaagtaagcataaatcaaatgattacagaatgggggaaaaaattaaacggtataaccgtacacgtcaccaaaacacaaccaccccactaaaacatcttactagttactagtatataagaatcatcaacgcactaagtaagacactcaacaaaacaaaacaagaaggagaatataagaagaagc

SEQ ID NO: 71 - DNA, 775 bp, Arabidopsis thaliana
  Drought-inducible promoter fragment from prAT5G43840 in Genbank AB026651, GI4757407
cgattttcgaataaattatttgagctttccaaactgtaattcaagtattattacttatatagtgttagtgtacttcaaaagttaaagcataaattttcttatatttgaaatgacctctttttacaaaatcttcttaaaattatgcattatcaatatattaattgtatatatatatataatgtataattctgcttgtgtcgtgcttaaccgtttgatttggtgtggttagatctggttttcccccaacccaattcaattgaatcaaggatcaatcaaattttcaaaggatactcttgttctctacacaaatctttcaaagggttccaccaaaaatcccatcattctgacttcagaataaacaaacaaaccacgaaacgtatctctatgcattcactacaacgtgtcatgggcgaaaacgaagcttataaatgttggagcatagtcactaaatttataatgattaattaaattttagattttctgatattcatagaagacaaaagaacacaaaagtagcatcttccaatgaatgtatgacactatgatctctcatttccatttatagcaaatcggctttgtccacatcaaagataactaataaatagacttatccaaaacactcaaaagcaatacatttctatccaaaaatattaaaccccaaaaatatagacagcataaaagcatcctcaagcttcagctattcatcacaactattctctcctctctctttttttattaaaaaagctcaaatttatataggttttttgttcacaaa

SEQ ID NO: 72 - DNA, 928 bp, Arabidopsis thaliana
  Drought-inducible promoter fragment from prAt5g66780 in Genbank AB010700, GI2828185
tagtcttgttgttttctcattagataatttaaactggtttgcttctttatttttggttggataaagtgaccggttctggtcatctgtttgagatgtaattactatttcataaaattaggaagttgaaagccaaatatatttgtaactactcttttatttgtaattttgctcaaaaaagtgatgaaatgtagttttgatatatgaatatctaccattatacataagtatatctgaacatggtacaacttatgaaagctaaatgtcaatacttgcaaagatataacaaatacaagttacatgaataagagatgtgtgttgaatttataagtgtcattttcttttcactttaaaacaaacttcatcttcttttgtttcttatgtgtcaaagttgccacagttgctctatttgagtctttcagtgtcagtctcagtcactgtactgattttacttttttttgttgagtgtgccaatgatgacatcactcccacgtcctccatccgtcttcttttaacggtcacgtggctcccaccctcttttctcgatgtctttaccgacttgttctagcccaacttacttgggccatttagattttttggtggcccaagttgctaaaagaggatttatcatagaaatctgaacccgttgcagcgctcaacacatgtcacagtcctgacaaacacgtattcaaatccttgttaagtcccgccacctgtcaccagagcaccacgaggcaaactctgatcaggacaccgtcgtactattatgtcggaagacaggaaagcttaattaagcttaaacctgacgtatttaacttcgttaactctaccttactaaagggttttaatttaaaacttatcatctcctcgtaagaataaaaactacttactctataaatttaagcttcaagaaacctccaaaagcagagaa

SEQ ID NO: 73 - DNA, 459 bp, Arabidopsis thaliana
  Drought-inducible promoter fragment from prAt3g17520 in Genbank AB022219, GI7321075
gggttttacttacaataagcccttactattcattgaaaagctcactaaacttgtttatgaaaagcccactggttattgtatacaagcccattagcttcacagatgtgtttcagttgaagctctctttgtttttgcgagtcggttttccgcaaaaagcaatcgcttgcctcgttgtttgtgtaacacgtgtcaagaaccacttaacacgaatccaaaatcgagaagccaaaagaagctggtactcgccacgtacttagccacgcgtcctaaacctatctctttttcaactaatacataacagagaagcaatcacagcaccattcctcggagaacacatcacagtaaacagaggtttttttcttcttctgaaacttgatataagttatataaccatataatattttgtgttcgattagtgtaacaaaaatggggttagagaggaaagtgtacggtttggtt

SEQ ID NO: 74 - DNA, 936 bp, Arabidopsis thaliana
  Drought-inducible promoter fragment from prAt4g09600 in Genbank AL161831, GI7321075
gttaaatcctcactaggatctctctttatattaatggttaaaaacatatgcatgttttgtgtttttgcatcttctttttcatagacaaaagcaagatgagtcttagaaggacatcaatgtcatagacatggctttagtatcttttgagtgtgctttaaatgatgatgatttaccctgaacctgaaattttacctattaattaatttaagtgtgcgttaaaccataaaccatatactctgaacctgaaattggttctaaagcacaacctaaacttgagattggagaatgctttaaaaggaaaaaaaaatcaaaggaaaccattaatgagccatcaaaaaatattcactaatatgacaagatgcattgtttatttttcttttcagaatcctcagaaactaccactaaactcctcaaggaacaaaaccatatcatgaattaggctggcaatttaactctgagacgtctttcttgtatagagaataaaacatacgcgtgtaaaagaaaacgcgtgaatcgaatgatgagtgttaacgttcgatcgagatgccaccaaatcttttcattaaaatgaattgtggaggacataccacttttaacgaggtcatttccactgggtgacatgtggactctactttgggtggcatgttcatatctttccacatcaccatgtaaacgtgaaaacacccaccacactcacttacatctcaaacacatgtcttcattatcgtacgtagctccaaaaaaaaaaatgaaaactaggtttagtgattctatttcgcaatgtataatatacaacttgtaaaaataaaatatttgaataagcattataaataaacccaaagaggtgttagatttatatacttaattgtagctactaaatagagaatcagagagaatagttttatatcttgcacgaaactgcatgctttttgagac